(19) United States
(12) Patent Application Publication
(10) Pub. No.: US 2007/0042373 A1
(43) Pub. Date: Feb. 22, 2007

(54) Protein identification methods and systems

(75) Inventors: Christopher Hogue, Toronto (CA); Jonathan Rose, Toronto (CA); Anish Alex, Toronto (CA)

Correspondence Address: HAMRE, SCHUMANN, MUELLER & LARSON, P.C., P.O. BOX 2902, MINNEAPOLIS, MN, 55402-0902, US

(73) Assignee: Mount Sinai Hospital, Toronto (CA)

(21) Appl. No.: 10/560,055

(22) PCT Filed: Jun. 4, 2004
(86) PCT No.: PCT/CA04/00853

§ 371 Date: Aug. 23, 2006

Related U.S. Application Data
(60) Provisional application No. 60/477,076, filed on Jun. 9, 2003.

(51) Int. Cl. C12Q 1/68; G06F 19/00

(52) U.S. Cl. 435/6; 702/20

(57) ABSTRACT

The present invention relates to methods and systems for identifying proteins. In particular the invention provides a method for identifying a protein through amino acid sequences of one or more query peptides generated from the protein. The method involves translating amino acid sequences of the query peptides to all possible codons from which the peptides can be synthesized to prepare strings of codons. Known nucleic acid sequences, in particular a set of known nucleic acid sequences including a genome, are searched to locate one or more known nucleic acids that comprise regions that match the strings of codons. Matching nucleic acids are ranked to identify nucleic acids that are true coding regions for the protein to thereby identify the protein.

### FIELD OF THE INVENTION

The invention relates to methods and systems for identifying proteins.

### BACKGROUND OF THE INVENTION

Database searching for peptide identification using mass spectrometry data as queries is now commonplace. However, an ongoing problem in mass spectrometry is the time it takes to search unannotated genomic DNA sequences with MS/MS peptide information, especially with large amounts of data as found in LC/MS/MS runs. Choudhary et al. (Proteomics 2001:651-667) reported the use of the genome as a database but the technique suffered from long search times. They reported search times of 10 hours on a single 600 MHz Intel CPU for 169 MS/MS spectra (about 3.5 minutes per spectrum). This is far longer than the acquisition time. Parallelization of any search software on a Beowulf cluster requires doubling the amount of computers each time to cut the search time in half. Thus, there is a need for fast and efficient methods and systems for identifying proteins from mass spectrometry peptide information.

The citation of any reference herein is not an admission that such reference is available as prior art to the instant invention

### SUMMARY OF THE INVENTION

The present inventors have developed a new approach to protein identification. The approach enables de novo protein sequencing of a genome in a very fast and cost effective manner. In particular, the multiple sequencing steps and final peptide ordering phase of conventional mass spectrometry sequencing methods can be avoided allowing the sequencing speeds and overall mass spectrometry throughput to be greatly increased. Using the methods of the invention only a few (e.g. 1, 2, 3) peptides from a protein need to be analyzed to obtain the full protein sequence. Thus, the methods and systems can use small quantities of proteins since only a few peptides need to be analyzed. In addition, the methods and systems by generating a list of peptide masses for the full protein sequence make it easier to distinguish true proteins in the sample and artifacts generated by noise from contaminant proteins.

In an aspect the approach utilizes mass spectrometric techniques and a hardware-based searching algorithm. This system is capable of locating peptide queries (interpreted mass spectrometry data) in a genome and scoring each matching location based on the uninterpreted data from the mass spectrometer.

Thus, the invention provides a method for identifying a protein through amino acid sequences of one or more query peptides generated from the protein comprising:
(a) translating amino acid sequences of one or more query peptides to all possible codons from which the peptides can be synthesized to prepare strings of codons;
(b) searching known nucleic acid sequences, in particular a set of known nucleic acid sequences including a genome, to locate one or more known nucleic acids that comprise regions that match the strings of codons; and
(c) optionally ranking two or more matching nucleic acids to identify nucleic acids that are true coding regions for the protein to thereby identify the protein.

In a particular aspect the invention provides a method for identifying a protein comprising:
(a) providing amino acid sequences of query peptides generated by mass spectrometry of peptides cleaved from the protein;
(b) translating amino acid sequences of one or more query peptides to all possible codons from which the peptides can be synthesized to prepare strings of codons;
(c) searching known nucleic acid sequences, in particular a set of known nucleic acid sequences including a genome, to locate one or more known nucleic acids that comprise regions that match the strings of codons; and
(d) optionally ranking two or more matching nucleic acids to identify nucleic acids that are true coding regions for the protein to thereby identify the protein.

In an embodiment of the invention, the strings of codons are provided as simultaneous parallel queries to a database of known nucleic acid sequences. In another embodiment, the nucleic acid sequences are also searched to locate nucleic acids sequences that comprise regions that match reverse complements of strings of codons.

In a still further embodiment, the method allows unknown amino acids in a sequence to be coded with a wildcard character. Thus, the strings of codons may optionally comprise wildcards.

In another embodiment of the invention, the ranking is based on a comparison of the masses of peptides translated from sequences in proximity to the regions in the known nucleic acids that match the strings of codons, with masses of peptides of the protein other than the query peptides.

In a particular embodiment of a method of the invention the ranking step comprises the following:
(a) calculating the masses of peptides translated from sequences in proximity to the regions in the known nucleic acids that match the strings of codons;
(b) comparing the masses calculated in (a) with masses of peptides of the protein other than the query peptides, or fragments thereof, to identify peptides with matching masses;
(c) assigning scores to each matching mass and accumulating the scores for all matching masses in proximity to the regions in the known nucleic acids that match the strings of codons; and
(d) optionally ranking two or more known nucleic acids that match the strings of codons based on the accumulated scores to identify potential nucleic acids encoding the protein to thereby identify the protein.

In an embodiment, the masses calculated in (a) are compared with masses identified by mass spectrometry for peptides of the protein other than the query peptides. In particular, the masses are compared with masses identified in a precursor ion scan (PIS).

The methods of the invention may involve further processing of the information concerning the potential nucleic acids encoding the protein. Such additional step may involve finding canonical splice variant masses that can be further compared with a PIS mass list to identify splice overlap peptides and help solve the gene structure of detected proteins.

In aspects of methods of the invention, the query peptides are at least 4 or 5 amino acids in length.

In another aspect of the invention, at least two query peptides are translated.

A method and/or system of the invention may generally comprise the following features:
(a) A method of locating potential coding genes within a genome. A database search engine is provided that is capable of locating query DNA strands within a genome.
(b) A method of translating genes to find the masses of tryptic peptides they generate. Once potential genes have been located, they are translated and digested in silico (by computation) to obtain the masses of the tryptic peptides.
(c) A method of comparing calculated tryptic peptide masses with masses detected by a first mass spectrometer. The tryptic peptides generated from each gene are compared with the precursor ion scan (PIS) list of masses. A scoring algorithm ranks every matching mass and thus a score for each gene match is generated to help the user to quickly identify the true coding gene.
(d) Fast overall processing time. Proteins should be identified in the time that a second mass spectrometer generates a sequence which on average is between 0.5 to 1 second.

The methods of the invention are generally executed in a computer apparatus/system.

In an embodiment, a computer implemented system is provided for identifying a protein through amino acid sequences of one or more query peptides generated from the protein comprising:
(a) a search engine for locating regions of known nucleic acid sequences that match strings of codons translated from one or more query peptides;
(b) a mass calculator for calculating masses of peptides translated from sequences in proximity to regions in known nucleic acid sequences that match the strings of codons; and
(c) optionally a scoring unit for (i) comparing masses calculated in (b) with masses of peptides of the protein other than the query peptides to identify peptides with matching masses; (ii) assigning scores to peptides with matching masses; and (iii) accumulating scores for all matching masses in proximity to or around the regions located in (a) to evaluate the likelihood that a region is a true coding region for the protein.

The invention further relates to a programmable hardware employing a method of the invention. In particular, a method of the invention may be implemented using a hardware acceleration system.

In an aspect the invention provides a hardware acceleration system for identification of a protein comprising a generic circuit board capable of being plugged into a computing device wherein the circuit board comprises logic chips and memory wherein the memory comprises nucleic acid sequence information, and the chips provide means to search through the nucleic acid sequence information for regions matching strings of codons translated from one or more query peptides provided to the computing device as input. The query peptide may be provided to the computing device as input from a mass spectrometer.

In an embodiment, a method of the invention is implemented using field programmable gate array (FPGA) technology. In another embodiment, a method of the invention is implemented using application-specific integrated circuit (ASIC) technology.

Information on the masses of the query peptides and peptides translated from the region around a hit or match nucleic acid sequence generated using a method of the invention and nucleic sequences and their scores identified using a method of the invention may be incorporated in or stored on a computer-readable medium or database. Thus, the invention provides a database storing data relating to strings of codons, matching nucleic acids, masses, scores, or methods of the invention. The invention also provides a computer system for storing this information.

The invention also provides computerized representations of information generated using a method of the invention, including any electronic, magnetic, or electromagnetic storage forms of the data needed to define it such that the data will be computer readable for purposes of display and/or manipulation.

The invention also contemplates a computer program product comprising a computer-usable medium having computer-readable program code embodied thereon for effecting the steps of a method of the invention, in particular identifying matching nucleic acids and identifying the protein within a computing system.

In an aspect the invention provides a computer comprising a machine-readable data storage medium comprising a data storage material encoded with machine readable data wherein said data comprises information generated using a method of the invention.

The invention also provides a system for managing and identifying proteins and methods for presenting information pertaining to nucleic acid sequences that potentially encode a protein.

Methods, systems, databases, and computer products of the present invention may be used to determine information for a protein. They may be used to identify protein sequences that, for example, may be associated with disease or that can be used in drug design. In an embodiment, the methods, and systems of the invention may be used to identify proteins in samples from patients.

These and other aspects, features, and advantages of the present invention should be apparent to those skilled in the art from the following drawings, detailed description, and example.

### DESCRIPTION OF THE DRAWINGS AND TABLES

The invention will now be described in relation to the drawings in which:

FIG. 1 shows a tryptic digestion of a large peptide.

FIG. 2 shows an outline of an algorithm of the invention.

FIG. 3 shows the architecture of a system of the invention.

FIG. 4 shows search engine amino acid and peptide units.

FIG. 5 illustrates locating a query in memory.

FIG. 6 shows parallel comparisons of identical queries to memory.

FIG. 7 shows a schematic diagram of calculator and detection units of a method and apparatus of the invention.

FIG. 8 shows a schematic diagram of calculator architecture of the invention.

FIG. 9 illustrates complementary strand calculations.

FIG. 10 is a schematic diagram showing a comparison of calculated masses with PIS.

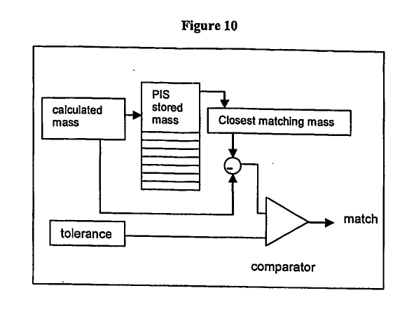

FIG. 11 is an example of a frequency table.

FIG. 12 is a schematic diagram showing architecture of a device of the invention.

FIG. 13 is a schematic diagram showing genome decompression.

FIG. 14 is a schematic diagram showing query reverse translation.

FIG. 15 is a schematic diagram showing full search engine architecture.

FIG. 16 is a schematic diagram showing a search of the genome.

FIG. 17 is a schematic diagram showing a peptide unit structure.

FIG. 18 is a schematic diagram showing a peptide unit operation.

FIG. 19 is a schematic diagram showing pipeline AND operation.

FIG. 20 is a schematic diagram showing a codon unit operation.

FIG. 21 is a schematic diagram showing implementation details of a codon unit.

FIG. 22 is a schematic diagram showing selection of a gene. Hit located in genome. Genes on either side of hit window are translated.

FIG. 23 is a schematic diagram showing translation of a gene to protein using a mass calculation process. Gene window translated from DNA to amino acid sequence.

FIG. 24 is a schematic diagram showing digestion of protein and calculation of tryptic peptide masses. Tryptic peptides detected in amino acid sequences. Peptide masses calculated.

FIG. 25 is a schematic diagram showing calculator architecture.

FIG. 26 is a schematic diagram showing a single stage of calculator.

FIG. 27 is a schematic diagram showing calculator subunits.

FIG. 28 is a schematic diagram showing complementary strand calculation.

FIG. 29 is a schematic diagram showing parallel six-frame calculations.

FIG. 30 is a schematic diagram showing scoring unit architecture.

FIG. 31 is a schematic diagram showing data associative mass storage.

FIG. 32 is a schematic diagram showing building the frequency histogram.

FIG. 33 is a schematic diagram showing updating of the histogram.

FIG. 34 is a schematic diagram showing mass matching.

FIG. 35 is a schematic diagram showing calculation of the product term.

FIG. 36 is a scaled representation of the distance between two queries.

FIG. 37 is a scaled representation of the distance between two queries.

FIG. 38 is a schematic diagram showing a device partitioned across TM3A

### DESCRIPTION OF EMBODIMENTS OF THE INVENTION

Given a mass spectrometry (MS) spectra of a peptide cleaved from a protein, it is possible to generate a corresponding sequence for the peptide (4). Since the peptide was cleaved from a protein, it can be assumed that there exists a gene within a genome that codes this protein. If the gene's coding region could be located quickly, it could be translated to its amino acid sequence. This longer sequence, obtained from the genome, can be compared to other fragments analyzed by mass spectometry as well as intron-exon splice variants.

Matching a mass spectrometry derived short peptide sequence to an unannotated genome represents an approach that Applicants have found to be well suited for hardware acceleration. Searching through unannotated DNA allows peptides to be identified that are missed by gene prediction algorithms as these are appreciable, even in organisms like Saccharomyces cerevisiae, expected to have a complete known set of protein coding regions (12).

As described herein the invention provides methods and systems for identifying a protein through amino acid sequences of one or more query peptides generated or cleaved from the protein. The methods and systems may be particularly useful for identifying proteins isolated from natural sources, patient samples, or from libraries that have been prepared synthetically.

The query peptides employed in the methods and systems of the invention may be generated or cleaved from a protein, in particular an unknown protein to be identified, using conventional techniques. In an aspect, peptides are generated using enzymatic digestion. In an embodiment, peptides are generated using proteolytic enzymes such as trypsin which cleaves at K and R residues (except where followed by proline).

The amino acid sequences of peptides generated from a protein may be determined using conventional molecular biology and recombinant DNA techniques and mass spectrometric techniques within the skill of the art.

In an aspect, the amino acid sequences of the peptides are determined using mass spectrometric techniques. In an embodiment, amino acid sequences of peptide fragments are determined using a tandem mass spectrometer. Examples of such devices include the MDS Sciex Q-Star, the Thermo Finnegan LCQ DECA XP, the MDS Sciex Q-TRAP, the Applied Biosystems TOF-TOF, the Waters/Micromass Q-TOF, the Bruker Daltonics APEX-Q, and other similar instruments capable of performing MS/MS. By way of illustration, a tandem mass spectrometer in a first stage performs a precursor ion scan (PIS) on tryptic peptides in a protein sample to provide an overview of tryptic fragment masses in the sample. The spectra obtained at this stage may be used to generate amino acid sequences for the peptides. In a second stage the mass spectrometer selectively filters peptides within a certain range into a chamber where the peptides are fragmented through collision with trace gases. In a third stage, the masses of collision-induced fragments are measured. The spectra obtained can be used to generate amino acid sequences for peptides.

The query peptides inputted into the methods and systems of the invention may be obtained from the spectra produced by the first or third stage of mass spectrometry. In an embodiment, the query peptides are obtained from the spectra produced by the third stage of mass spectrometry.

An amino acid sequence of a peptide is translated to all possible codons from which the peptide could have been synthesized to prepare strings of codons. There may be multiple codons for each amino acid in the peptide. In an embodiment, reverse complements of every query condon string are generated and searched against the known sequences. In another embodiment of the invention a computer is utilized to translate an amino acid sequence to all possible codons that it could originate from. The information may be converted to a form that allows for compression of the strings of codons. In an embodiment, the information is converted to a 3-bit encoded form that utilizes wildcards.

Known nucleic acids or sequences, particularly a set or database of known nucleic acids or sequences are searched to find regions of known nucleic acids that match strings of codons. Known nucleic acids or sequences include nucleic acid sequences from an organism, in particular an organism whose entire DNA is sequenced. The whole genomes of many organisms are reported by the NCBI at www.ncbi.nlm.nih.gov/entrez/query.fcgi?db=Genome, and in the scientific literature. In an embodiment, the known nucleic acid sequences comprise the human genome or multitude of human genomes. In another embodiment, the known nucleic acid sequences comprise a set or database or unannotated genomic DNA sequences.

In an aspect of the invention, the search of known nucleic acid sequences may be accomplished by aligning multiple copies of the query string of codons with successive positions within known nucleic acid sequences. In another aspect, the search may be accomplished by comparing strings of codons to known amino acid sequences, reading a new base into the known nucleic acid sequences, and shifting the nucleic acid sequences over by one position.

If multiple hits or matches are located in known nucleic acid sequences each is ranked according to its likelihood of being the true coding region. Ranking may be achieved by selecting nucleic acid sequences in proximity to or around (in particular, on either side of) a known matched region, translating the sequences into peptides and corresponding peptide (e.g. tryptic) fragments, and determining the mass of each of the fragments. In an embodiment, gene-sized windows of nucleic acid sequence are selected on either side of a matched region (e.g. 10 Kbases). Masses of peptides are determined sequentially until a breakpoint is reached. Breakpoints may be defined as a codon that indicates a proteolytic enzyme cut site (e.g. K or R if not followed by P for trypsin) or a STOP codon.

The calculated masses are compared with masses of peptides of the protein to be identified other than the query peptides. The calculated masses may be compared to the masses seen in a precursor ion scan (PIS) of the peptides, other than the query peptides, generated or cleaved from the protein to be identified. A score is assigned to each match based on a comparison of the masses of adjacent peptides in the matched known sequence with the adjacent sequences in the unknown protein. The match scoring system can incorporate both the frequency of occurrence of individual peptides and the number of matches in the final score. The match scoring system can incorporate both the frequency of occurrence of individual peptides and the number of matches in the final score. Matching masses can be determined within a predetermined threshold (e.g. <1 Da). The threshold may be used to identify standard amino acid variants (e.g. oxidized states or translational modifications). A scoring function may be used to rank matching peptides.

In a computer implemented method, a mass calculator is used to translate all frames simultaneously and produce the masses of fragments in parallel with the search of known nucleic acid sequences.

The methods of the invention are preferably executed in a computer apparatus/system. Included in a particular system of the invention is a processor comprising a mass calculator and scoring functions coupled to databases of known nucleic acid sequences, and various input/output devices such as a keyboard, mouse, display monitor, printer, and the like. A processor may be of the PC or standalone type, and have processing capabilities of at least an Intel Pentium I processing chip. Other processors such as a minicomputer, parallel processor, or a networked computer may be suitable.

In an aspect of the invention a computer implemented system is provided for identifying a protein through amino acid sequences of one or more query peptides generated from the protein comprising:
(a) a search engine for locating regions of known nucleic acid sequences (e.g. in a database) that match strings of codons translated from one or more query peptides;
(b) a mass calculator for calculating masses of peptides translated from sequences in proximity to regions on known nucleic acid sequences that match the strings of codons; and
(c) optionally a scoring unit for (i) comparing masses calculated in (b) with masses of peptides of the protein other than the query peptides to identify peptides with matching masses; (ii) assigning scores to peptides with matching masses; and (iii) accumulating scores for all matching masses in proximity to or around the regions located in (a) to evaluate the likelihood that a region is a true coding region for the protein.

In an embodiment, the computer implemented system comprises more than one mass calculator with each calculator operating in parallel to produce multiple output masses. Additional mass calculators may compute masses of each frame and its complement. In another embodiment, multiple instances of the scoring unit are implemented, one for each output of the mass calculator.

The invention particularly contemplates a hardware accelerator system or programmable hardware for executing a method of the invention. In particular, a method of the invention may be implemented using a hardware acceleration system.

In an aspect the invention provides a hardware acceleration system for identification of a protein comprising a generic circuit board capable of being plugged into a computing device wherein the circuit board comprises logic chips and memory wherein the memory comprises nucleic acid sequence information, and the chips provide means to search through the nucleic acid sequence information for regions matching strings of codons translated from one or more query peptides provided to the computing device as input.

In an aspect the invention provides a hardware acceleration system for identification of a protein comprising a generic circuit board capable of being plugged into a computing device wherein the circuit board comprises logic chips and memory wherein the memory comprises nucleic acid sequence information, and the chips provide means to search through the nucleic acid sequence information for patterns matching a query that has been provided to the computing device as input from a mass spectrometer.

In the systems of the invention the circuit board has access to the host computing device's memory with its operation being controlled by the host.

In an aspect, a method of the invention is implemented using field programmable gate array (FPGA) technology. In another aspect, a method of the invention is implemented using application-specific integrated circuit-(ASIC) technology.

In an embodiment, the invention provides a computer system comprising one or more field programmable gate array (FPGA) logic chips together with memory storage and input and output channels which communicate to a computing device, wherein the memory holds nucleic acid sequence information, and the FPGA logic chip initiates searches through the nucleic acid sequence information for matching strings of codons translated from one or more query peptides provided to the computing device as input.

In a particular embodiment, the FPGA logic chips in a computer system of the invention comprise a search engine, one or more mass calculators, and one or more scoring units. Thus, the invention provides FPGA logic chips in a computer system comprising
(a) a search engine for locating regions of known nucleic acid sequences (e.g. in a database) that match strings of codons translated from one or more query peptides;
(b) one or more mass calculators for calculating masses of peptides translated from sequences in proximity to regions on known nucleic acid sequences that match the strings of codons; and
(c) one or more scoring unit for (i) comparing masses calculated in (b) with masses of peptides of the protein other than the query peptides to identify peptides with matching masses; (ii) assigning scores to peptides with matching masses; and (iii) accumulating scores for all matching masses in proximity to or around the regions located in (a) to evaluate the likelihood that a region is a true coding region for the protein.

In another embodiment, the invention provides a computer system comprising one or more field programmable gate array (FPGA) logic chips together with memory storage and input and output channels which communicate to a computing device, wherein the memory holds nucleic acid sequence information, and the FPGA logic chip initiates searches through the nucleic acid sequence information for matching data that has been provided to the computing device as input which has originated from a mass spectrometer.

In an embodiment, the system performs a six frame translation word search with wildcards.

In a particular embodiment, a computer system of the invention is capable of search speeds of about 500-800 MB/s or about 1.5-2 Gbases. It will be appreciated by a person skilled in the art that the speeds may be improved, in particular, with faster FPGAs or ASICs.

In a particular embodiment, the nucleic acids are encoded in a 3-bit encoding (A=000, T=001, C=010, G=100 and N=100 for ambiguities). In another particular embodiment, the hardware acceleration system or FPGA comprises logic that performs a calculation estimating the masses of peptide fragments in proximity to or around each region of a matching nucleic acid sequence found by the search. In yet another embodiment, the masses of peptides are scored using logic that counts the frequencies of such masses and computes a score proportional to the likelihood that each fragment is represented in mass data provided to the computer device as input by the mass spectrometer. In still another embodiment, the system or computing device returns as output, the location of each match in the known nucleic acid sequences, and the score that the match represents in the observed sample. In a particular embodiment, the output comprises the score that the match represents in a sample observed in a mass spectrometer.

In a specific embodiment of the invention the programmable hardware is a Transmogrifier 3A (TM3A) reconfigurable platform (11) with Virtex II 8000, Stratix S40, and/or Stratix S80 FPGA chips that are interconnected to each other. Each FPGA can have SRAM attached and various 10 connectors. Data can be read from the SRAM in 63-bit words. Bach chip can be connected to a central housekeeping chip which performs the configuration of the FPGAs and ensures that they are functioning within their operational limits. The housekeeping chip also interfaces the board with a PC. The PC allows the user to download designs into the onboard FPGAs and to communicate with the board to provide input and receive output.

Information generated using a method of the invention, including strings of codons derived from query peptides and complements thereof, the masses of the query peptides and peptides translated from the region around a hit or match nucleic acid sequence, and the identity of the matching known nucleic sequences, their scores and ranking, may be incorporated in or stored on a computer-readable medium or database. Thus, the invention provides a database storing data relating to strings of codons, matching nucleic acids, masses, scores, or methods of the invention. The invention also provides a computer system for storing this information.

In an embodiment, the invention contemplates a database comprising a set of masses corresponding to the masses of the query peptides and the peptides translated from a matching region in proximity to or around a known nucleic acid generated in a method of the invention. The invention also contemplates a database comprising scores assigned to peptides with matching masses, accumulated scores for all matching masses, and nucleic acid sequences identified using a method of the invention that potentially encode a protein to be identified and the scores and rankings for the nucleic acids.

The invention also provides computerized representations of information generated using a method of the invention, including any electronic, magnetic, or electromagnetic storage forms of the data needed to define it such that the data will be computer readable for purposes of display and/or manipulation.

In an aspect the invention provides a computer comprising a machine-readable data storage medium comprising a data storage material encoded with machine readable data wherein said data comprises information generated using a method of the invention.

The invention also provides a method for presenting information pertaining to nucleic acids that potentially encode a protein the method comprising the steps of: (a) providing an interface for entering query information generated from mass spectrometry relating to amino acid sequences of peptides generated or cleaved from the protein; (b) examining records in a database of known nucleic acid sequences to locate regions in the nucleic acid sequences matching strings of codons translated from the entered query peptides' amino acid sequence information; (c) displaying the data relating to the matched string of codons and regions in the nucleic acids; and (d) optionally displaying the masses of the peptides generated from mass spectrometry and the masses of peptides encoding regions in proximity to the regions of known nucleic acids that match the string of codons. The method may also comprise displaying scores for each matching mass, accumulated scores for all matching masses around or in proximity to the regions, and/or the rankings for the nucleic acids based on the accumulated scores.

The invention also contemplates a computer program product comprising a computer-usable medium having computer-readable program code embodied thereon for effecting the steps of a method of the invention, in particular, identifying matching nucleic acids and identifying the protein within a computing system.

The invention also provides a system for electronically identifying proteins employing a genome of an organism.

Methods, systems, databases, and computer products of the present invention may be used to determine information for a protein. They may be used to identify protein sequences that, for example, may be associated with disease or that can be used in drug design. In an embodiment, the methods and systems of the invention may be used to identify proteins in samples from patients.

Having now described the invention, the same will be more readily understood through reference to the following examples that are provided by way of illustration, and are not intended to be limiting of the present invention.

### EXAMPLE 1

In-Silico Search Strategy

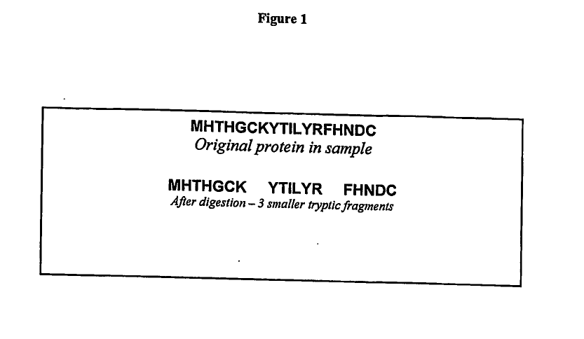

A protein sample can be prepared for mass spectrometric analysis by standard techniques (8). If a specific proteolytic enzyme such as trypsin is used the peptide will be cleaved at its K and R residues (except where followed by Proline). This process is illustrated in FIG. 1.

These peptide fragments are introduced into the first stage of a tandem mass spectrometer through a variety of techniques (9) (10). There are generally three stages of MS/MS operations. In the first stage, the mass spectrometer performs what is known as the precursor ion scan (PIS). The PIS gives an overview of the tryptic fragment masses in the sample. In the next stage, the MS can then act as a filter to selectively pass fragments within a certain range into the next chamber. Here the tryptic peptides are allowed to fragment through collision with trace gases (e.g. N₂). The next chamber is used to accurately measure the mass of collision-induced fragments, which are selected individually from the second chamber, usually in order of abundance. This last stage can be time consuming if there are many fragments. Mass spectrometry measurements consume the sample, so if the sample is small, it may run out before each of the fragment masses found in the PIS can be processed all the way to the third stage. This is especially true for systems employing small volume liquid separation methods to introduce sample into the instrument.

Using the conventional techniques for analysis (5) the spectrum obtained from this stage can then be used to generate a peptide sequence, but will fail to sequence peptides that either do not exist in the protein database or those peptides that occur as a result of nucleotide polymorphisms.

Using the hardware accelerated search system described herein, the individual steps of the MS process are not modified in any way. However, following the first sequencing, it may not be necessary to process all fragments in the PIS stage using MS/MS techniques. If one can quickly locate the gene of origin in unannotated genomic DNA sequence for the first tryptic peptide fragment that has been sequenced by the MS/MS stage, then one can infer the masses of other tryptic peptides arising from the same gene, and identify them from the list of masses at the PIS stage directly. This strategy effectively reduces the database search size to a gene-sized window setting for pursuing a statistical scoring scheme using PIS mass information detailed herein.

To this end, all possible DNA sequences that could have coded this fragment are generated (i.e. the peptide query is reverse translated from amino acids into strings of all possible codons). This is quite different from conventional approaches that apply 6-frame translation to the database and search in amino acid sequence space. There may be multiple codons for each of the amino acids in the short subsequence of the tryptic fragment as detected, and thus multiple query DNA sequences to search for. Intuitively, this requires a wildcard query approach.

It is also likely that there will be several matches in the human genome for the query sequences, since they are relatively short in length and may exist in many proteins. To resolve which of these hits actually is the unknown protein, a section of the DNA surrounding each of the hits is taken into consideration. The section will encompass approximately a gene-sized window on either side of the hit. This section is immediately reverse-translated in silico to determine what coding regions it contains. Since the original sample was trypsin digested, the same procedure is applied to the translated sequence (i.e. it is split into several peptide fragments at its K and R boundaries, excluding KP and RP). The mass of each of these smaller fragments is then determined and compared against the list of masses detected by the PIS. If the two masses match within some specified tolerance, a sequence is assigned, and aggregate statistics can be used to determine the likelihood of the correspondence of individual tryptic fragment matches between the PIS data and the gene found.

Algorithm Overview

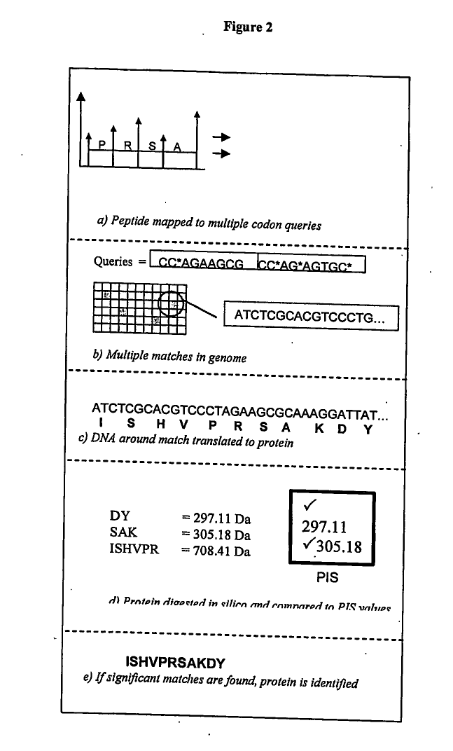

A peptide query can be obtained from the spectra produced by the third stage of the MS (4). Each amino acid in this query sequence is translated to the possible codons that it could have originated from (one such example shown in FIG. 2 a). Each of these potential codon strings is provided as a simultaneous parallel query to the human genome database. Any locations in the genome which contain these coding regions are flagged (FIG. 2 b).

If there are multiple coding regions, the DNA sequence on either side of the hit location is considered. A gene-sized window of DNA (10 Kbases in the current implementation) is selected on either side of the hit and translated into its peptide and corresponding tryptic fragments (FIG. 2 c). The mass of each of these fragments is then compared to the list of masses generated by the PIS. If there are masses that match within some user-defined threshold (usually <1 Da), aggregate statistics for each match are recorded (FIG. 2 d). Based on the MOWSE scoring algorithm (6), matching peptides are ranked based on their frequency of occurrence. The score for each hit then corresponds to the likelihood that peptide matches are random.

The basic flow of the algorithm can be summarized as follows: Each possible coding region of a query peptide is identified and returned along with score indicating the likelihood that this region is the true coding sequence. There are several advantages to this approach. Firstly, the last stage of the MS described above may not need to be repeated several times. Furthermore the final step of ordering individually sequenced peptide fragments can also be eliminated. Software implementations of similar methods tested have no capacity for wildcard expansion and are not fast enough for high-throughput protein sequencing as the genome search takes approximately 3.5 minutes per spectrum on a 600 MHz Pentium processor, which would be expected to scale to only 52 seconds on an Intel 2.4 GHz processor commonly available on PCs.

Hardware FPGA Implementation

To leverage the advantages of the solution described herein and obtain real-time performance, a hardware FPGA implementation of the process outlined herein has been built. Three key components are required: Primarily, a search engine is needed to locate the possible coding regions of the peptide within the genome. A mass calculator is needed to produce the masses of tryptic fragments in the gene window surrounding all potential match locations. Lastly, a means of evaluating the likelihood that a given location in the genome is the true coding region for an unknown protein is required. The scoring unit compares the masses generated by the calculator to those found in the PIS, and ranks hit locations based on the quality of the match.

Implementation

The approach described herein has been prototyped on the University of Toronto's Transmogrifier 3 (TM3) hardware platform. The TM3 is a prototyping board with four interconnected Xilinx Virtex 2000E FPGAs, onboard RAM and various 10 connectors to allow the addition of peripheral devices. It also has a software interface that allows it to communicate with a host PC. It allows for search speeds of 700 MB/s or approximately 1.9 Gbases/s.

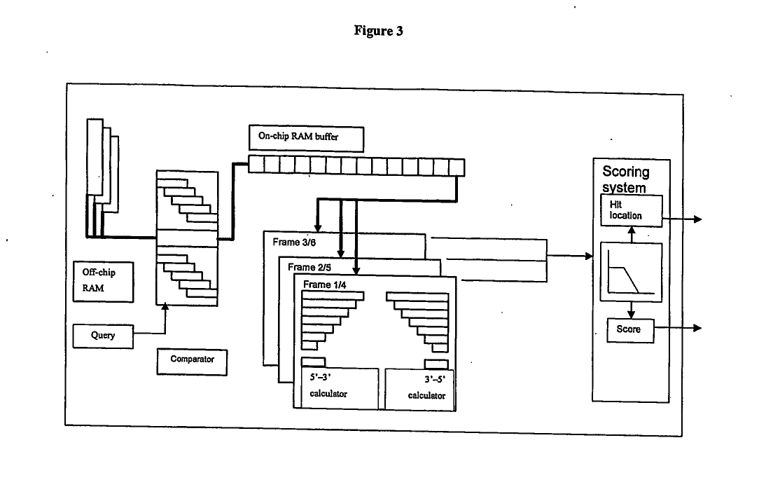

To implement the above algorithm, the onboard RAM is loaded with the genome, and the FPGAs are loaded with the search engine, mass calculator and scoring unit. The device is initialized by sending in the peptide query followed by the list of masses detected in the PIS. All locations in the genome which could possibly have coded the query peptide were first searched and then the masses of the surrounding tryptic fragments were calculated. If a significant number of matches are found between the calculated fragment masses and the PIS, a likely coding region has been found. The host PC then receives a list of all locations at which the query was found along with the score indicating the quality of each of these matches. The general flow is depicted in FIG. 3.

Human Genome Database

The genomic database sequence is loaded into the onboard RAM on the TM3 when the device is initialized. It is stored in a 3-bit encoding which allows for eight possible characters, five of which are used (A=000, T=001, C=010, G=100 and N=100 for ambiguities). Substantially more RAM may be used on board to store the entire human genome. The encoded database is obtained from a FASTA file, which is translated from its text form into the 3 bit version in software. When the device is initialized, the encoded database is loaded into the off-chip RAM surrounding the FPGA.

Search Engine

As described herein, the primary objective of the algorithm is to identify all possible locations in the genome from which a peptide may have originated. To accomplish this, the user provides a peptide query (inferred from the spectra generated by the last stage of the MS), which is simply a string of amino acids. Each amino acid in the string is translated (in software) to the codons from which it may have been synthesized with optional wildcards. These strings of codons are converted to the 3-bit encoding described herein and sent to the search engine. Thus the query entering the search engine is no longer in amino acid form, but rather a set of all possible DNA strands. The search engine will report the locations within the genome in which a query string is found.

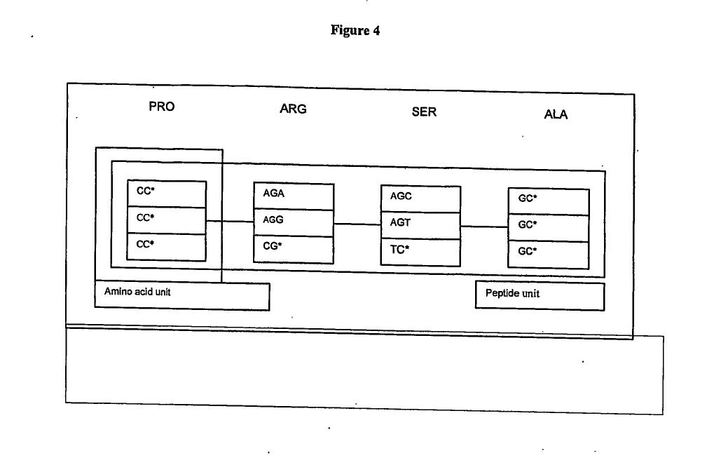

One of the advantages of the 3 bit encoding is that it allows for compression of the query string. Consider for example the amino acid query: Pro-Arg-Ser-Ala. There are six possible codons for Arg and Ser (FIG. 4). They can be compressed to two unique codons and one codon with a wildcard on the wobble-base. Thus, each amino acid can be encoded into three codon registers or less. Note that FIG. 4 implies that there is a hierarchy of units within the search engine. At the lowest level, there is an amino acid unit, which accepts potential query codons (from the MS) and memory codons (from the genome) as inputs. If any of the query codons match a memory codon the amino acid unit indicates a hit. Only a single comparison is needed to test all potential codons in a single unit against a single memory codon.

The next level of hierarchy is the peptide unit, which consists of several (10 in this implementation) amino acid units. If all of the amino acid units in a peptide unit indicate a match, a memory string corresponding to the query has been found. This is apparent from the structure shown above, as it implies that a sequence of memory can be grouped into codons that translate into the query amino acid sequence. For the example above, if the memory string CCC AGG TCA GCA was read in from memory it would produce a match with the peptide unit shown above.

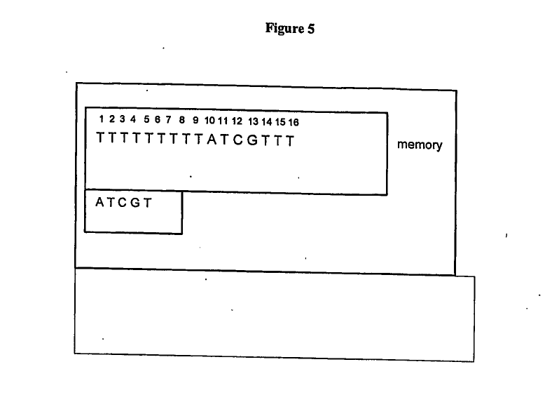

Once initialized, the search engine reads in the genome from the RAM and starts comparing it to the query strings as shown above. One approach is to compare a memory string to the query strings and then read a new base into the memory string and slide it over by one position. In this manner, the search engine moves through the entire genome database and compares it against all possible query strings. Consider the example shown in FIG. 5. A match to the query string clearly exists in the database string. However, to discover this match, the memory string must be shifted by nine bases. In the naïve implementation described above, this would be accomplished by multiple serial comparisons as nine bases are shifted in. A better implementation would have multiple copies of the query aligned with successive positions within the memory string. In FIG. 5, if there were 10 copies of the query, the first aligned with position one (as above), the second with position two and so on, the 10^{th} copy would detect a match with the memory string.

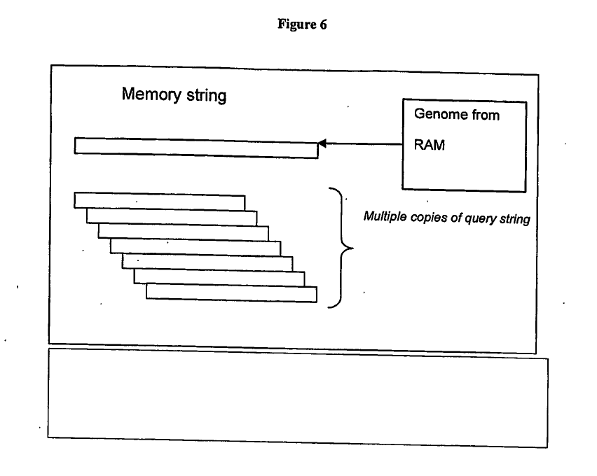

To operate at the full memory bandwidth of the TM3 (1 memory word per cycle) all these comparisons have to take place in a single cycle. In the hardware, multiple copies of the query register are implemented, one for each position in the memory string. A depiction of the query registers aligned against the data from memory is provided in FIG. 6. All comparisons occur in parallel therefore the query is simultaneously compared to each subsequent character position in the memory string.

If any of the positions match, a hit to the current genome (memory) address is recorded. Note also that the copies of the queries are staggered at one-base intervals instead of one-codon intervals. Due to this approach, the three 5′-3′ reading frames are automatically considered. Note that the DNA sequences in the genome are unidirectional. There is only a 5′-3′ or 3′-5′ copy of any given sequence in the genome file. To account for this, the reverse complement of every query strand is also added as a query to the search engine. The complement is also staggered in the manner depicted by FIG. 6 which automatically covers the three 3′-5′ reading frames. All complementary strand reading frame comparisons occur in parallel therefore the query is simultaneously compared to each subsequent character position in the memory string.

With this approach, a single peptide query is converted to all potential coding sequences and their reverse complements, and the search engine will find any locations that contain these strands. Each hit location must then be evaluated by checking if the MS discovered any tryptic fragments surrounding the hit.

Cutsite Detection and Mass Calculation

In general FPGA implementations are not efficient in dealing with floating-point arithmetic. Therefore all calculations carried out are done with 20 bits shifted by a constant factor of 102 effectively allowing 2 decimals of precision. These can be modified to increase precision with slight area and speed penalties on the current hardware.

Once the search engine has located a match to the query, the significance of the match must be determined. As mentioned earlier, there may be multiple matches to a short peptide query and it remains to determine which of these matches is the true coding gene. To this end the masses of tryptic fragments are calculated around every match. If a certain hit location has several neighboring tryptic fragments that correspond to the masses found in the PIS, it is likely that this hit location codes the protein in the sample.

To obtain the mass of fragments surrounding the hit, the genome, is passed through a shift register, which acts as a buffer. The shift register delays the RAM words and keeps them in the device. When a hit is detected, the calculator begins accepting words from this buffer; if the buffer is the size of a gene, calculations effectively begin at one gene window size preceding the hit location and end calculations after one gene window size following the hit location. In an implementation the size of a gene is assumed to be 10K bases and therefore tryptic masses are calculated for a 20K base “window” around the hit location.

The calculation of tryptic masses is straightforward. Each codon in the genome is translated to its corresponding amino-acid mass. These masses are accumulated sequentially until a breakpoint is reached. The breakpoint can be any codon that indicates a tryptic cut site (K or R if not followed by P) or a STOP codon. Once a breakpoint is encountered, the accumulated mass, corresponding to a single tryptic fragment, is forwarded to the scoring unit for comparison with the PIS list. Once again, in a naïve implementation, each tryptic fragment would be sequentially analyzed and its mass would then be scored. However the device is pipelined to match the throughput of the search engine (1 memory word/cycle). As a result, the calculator consists of several processing units that operate in parallel. In a 63-bit memory word there are 21 bases or 7 codons. Correspondingly the calculator has a 7-stage pipeline to calculate the masses of the seven codons in parallel.

The first stage will buffer the first 63-bit memory word, but only calculate the mass of the amino acid created by the first codon in the current memory word. It will also determine if the codon indicates a cut-site. In the next cycle, the first stage will receive a new 63-bit word as its input and will pass the mass and cut-site information to the second stage, along with all the remaining codons in the first word. The second stage will then add the mass of the received codon to the mass of the second codon in the first memory word, which it calculates. This accumulated mass will then be passed to the next stage along with the cut-site information and the remaining codons. The process is repeated for each stage and the masses of several tryptic fragments are calculated in parallel.

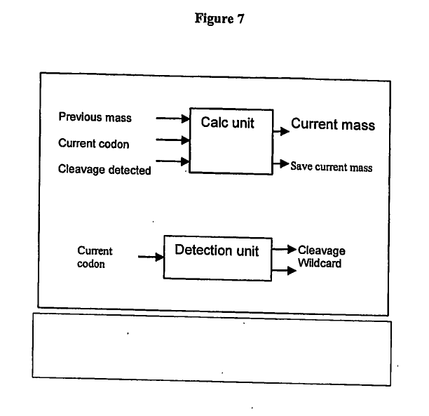

At each stage there is a calculator unit that receives the masses of the previous codon and the current codon. It also receives information about whether a tryptic cut site or cleavage point was detected in the previous stage. These data allow the current stage to calculate new masses and determine whether it should save them. There is also a detection unit that looks for cleavage points and wildcards in the codons. The wildcard is represented by the ‘N’ or ambiguity character, which may exist in the genome. If a cut site is detected the current mass is saved. If a wildcard in the codon creates an irresolvable amino acid, the mass is discarded. The calculator and detection units are depicted in FIG. 7.

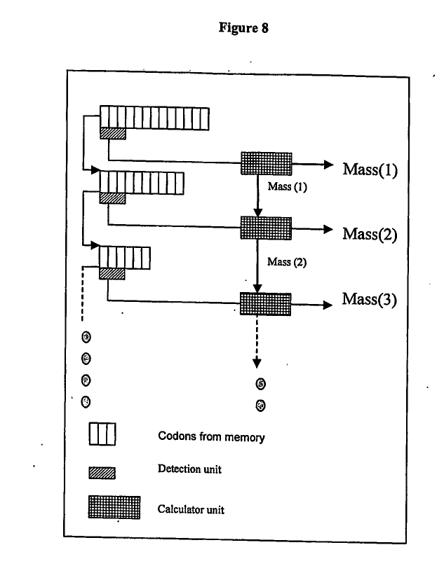

Each stage of the calculator has a calculator unit and a detection unit. With the aid of a central controller each unit outputs masses to be saved and discards masses that cannot be resolved. In every cycle a new memory word is read in and its first codon is processed in the first stage. In the next cycle a new memory word is read in and the remainder of the old word is passed to the next stage where its next codon is processed as described. The overall architecture of the calculator is illustrated in FIG. 8.

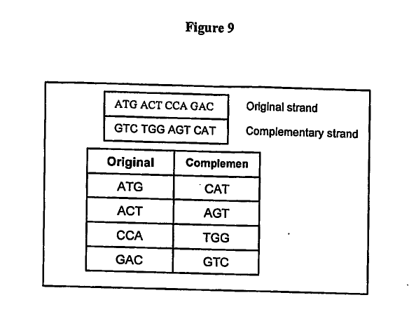

As with the search engine, the complementary DNA strand must be accounted for. The tryptic masses for both the strand stored in the genome and the reverse complement that it implies must be calculated. With the hardware above, the masses of tryptic fragments from the original strand can be calculated. For the complement, a copy of this hardware is built which transposes and complements the codons as required for the complement. In FIG. 9 an example string is shown alongside its reverse complement. Note that to obtain the reverse complement the original strand is transposed and the bases are replaced with their complements. However, the codons arriving from memory arrive in the order of the original strand. As shown in FIG. 9, the codons are accumulated in the forward direction for the original strand, but backwards for the complementary strand. This obviously has no effect on the accumulation of tryptic masses, which is an associative operation.

Each calculator unit computes the masses of one strand and its complement. This accounts for one frame and its complement. To account for the other two frames and their complements, two more calculator units are instantiated; each starts at one base position ahead of its predecessor. This is depicted in FIG. 3 as three calculators operating in parallel. All masses are calculated with 20-bit precision and the stored values for each amino acid mass are accurate to within 1/100^{th} Da.

Scoring Unit

When multiple hits are discovered for a query, each hit can be optionally ranked relative to the others through the addition of the scoring unit to the searching system. To do this the masses of tryptic fragments around each hit are compared to those detected in the PIS. If the tryptic fragments around a given hit match those detected by the PIS, it is very likely that the hit corresponds to the true coding sequence.

The scoring unit is used to provide a ranking of the gene windows. If multiple hits (windows) are detected, only a few of them may be the true coding region for the sample in the MS. The score can be used to quickly evaluate which window is the most likely coding region.

There are two stages to the scoring algorithm. Firstly, a calculated mass must be compared to masses detected by the PIS. Once the closest match in the PIS is found, the difference between the two masses is computed. If this difference is within a user-defined threshold, a match is indicated. These thresholds can also be used to consider standard amino acid mass variants such as oxidized states or translational modifications. The second step is to assign a score to each of the matching masses. The score is used to evaluate the likelihood that a given match is not random. Scores are generated using techniques similar to those used by MOWSE (6) and rely on the assumption that for a true match, a statistically improbable number of matches are observed within a gene sized window to masses accumulated in the PIS.

Upon initialization, the PIS masses are sent to the scoring unit, which saves them in on-chip RAM. When the masses from the calculator are generated, each must be compared with the stored masses from the PIS. This process corresponds to the first step described above.

A data-associative indexing scheme is used to facilitate rapid comparisons. The on-chip RAM can essentially be thought of as a set of mass bins. Masses falling into a certain range are stored in the bin corresponding to that range. Consider for example RAM of depth 2048—there are 2048 unique storage locations (mass bins/mass ranges) available. Masses can range between 0 and 10485.75 Da, since they are 20 bit values (2²⁰=1048576) and all floating point numbers are treated as integers shifted by two decimal places. In the data associative storage scheme, the 11 (2¹¹=2048) most significant bits (bits 19 to 9) of the mass are used as an address at which to store the mass. Note however, that in a 20 bit mass this implies that masses to be stored must be greater than 5.12 Da apart since there are 9 non-address bits (bits 8 to 0) (2⁹=512). This is a constraint on the values in the PIS. It can be modified by adding more storage (i.e. more than 2048 locations) but this will result in greater area usage on chip. Mass fragments generated by the calculator are then used as addresses to retrieve their closest matching PIS values. The difference between the calculated mass and the stored PIS mass is then calculated. If it meets a user-defined threshold (between 0 and 1 Da), the current calculated mass is flagged as a match.

For this matching mass a score must then be calculated. This is done using a technique similar to that used to calculate the MOWSE factor matrix M. The M matrix has as its elements

$${m}_{i , j} = \frac{{f}_{i , j}}{\vert {f}_{i , j ( max )} \vert}$$

where f is an element of the frequency factor matrix F.

The frequency factor matrix is a histogram of frequencies spanning the observed peptide mass range over the gene-sized window. The MOWSE factor matrix M then, is simply a normalized representation of these values. The frequency factor matrix F has columns that represent intervals of intact protein mass. More importantly, each individual column has several rows which represent 100 Da intervals in peptide mass. As peptide masses are generated, the appropriate row is incremented to keep track of how frequently masses fall within a certain range. When matching masses are found, a score is generated for each entry based on the formula:

$$Score = \frac{50000}{( {M}_{prot} \times \prod {m}_{i , j} )}$$

Where Mₚᵣₒₜ is the molecular weight of the protein in the traditional MOWSE search. Since the implementation does not utilize intact proteins the following representation is used as the score for a window.

$$Score = \frac{K}{( \prod {m}_{i , j} )}$$

where K is a scaling factor that can be set by the user.

The scoring algorithm calculates all rows of the frequency factor matrix (one column) for individual gene windows and then calculates the score using the formula above.

Note also:

$$\begin{matrix}\prod {m}_{i , j} = \frac{{f}_{1}}{{f}_{max}} \times \frac{{f}_{2}}{{f}_{max}} ⋯ \frac{{f}_{m}}{{f}_{max}} \\ = \frac{\prod f}{{( {f}_{max} )}^{m}} where m is the number of matches .\end{matrix}$$

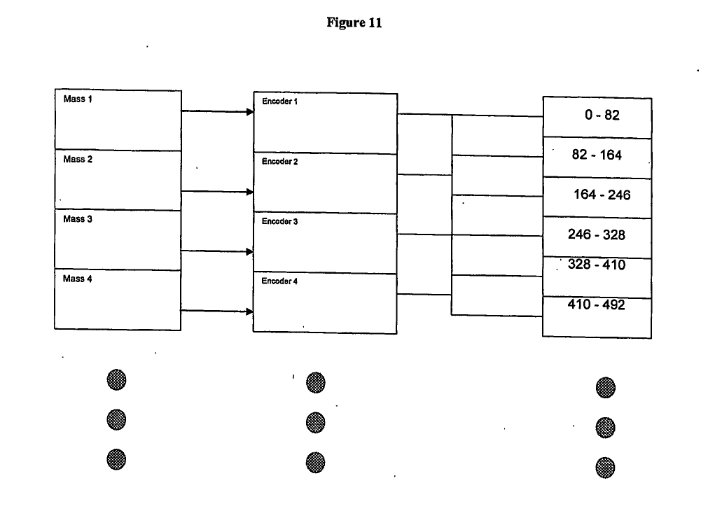

The evaluator consists of a frequency table with 128 sets of 82 Da bins (FIG. 11). These represent the rows of the F matrix. Each new mass that is computed is passed through the frequency table that keeps track of which bin the mass falls into. Note that this relies on the assumption that masses will fall into the 20-bit range. The allowable masses are between 0 Da and 10485.75 Da. The 128 bins require 7 bits to index them. These are the 7 most significant bits of the mass (bits 19-13), and thus will divide the bins into 81.9 Da ranges. Once all the masses in a window have been considered the frequency table will have a count of how many tryptic fragments fall into each different range.

To calculate the product term, each calculated mass is once again passed through the frequency table. In this pass the table already has the frequency with which a mass in this range was detected in the current gene window. This frequency is passed to a logarithm unit which calculates log₁₀(frequency of current matching mass). The use of logarithms allows a larger range of numbers to be represented and avoids the speed and complexity requirements of hardware multipliers. The logarithms are calculated in hardware by lookup tables since only the logs of integer values over a relatively small finite range are required. These logarithms can then be added together to obtain the product term.

$${\prod }_{i = 1}^{m} {f}_{i} = {\sum }_{i = 1}^{m} log ( {f}_{i} )$$

This product term, along with the maximum frequency and number of matches is returned to the PC to calculate the frequency given by.

$$Score = \frac{K}{\frac{{\prod }_{i = 1}^{m} {f}_{i}}{{( {f}_{i , j} max )}^{m}}}$$

The calculator is capable of simultaneously producing eight masses, therefore, to use the frequency table, each of the calculator outputs must be considered simultaneously. In FIG. 11 each calculator output is passed into an encoder that determines which range it falls into as described above. The frequency table monitors the outputs of each encoder and increments the referenced bins by the number of encoders that refer to it.

If the stored mass matches the calculated mass within a user specified tolerance, the scoring unit increments the number of matches.

Observe in FIG. 8, that the calculator produces multiple output masses. To ensure that each of these masses is included in the scoring of a hit location, multiple instances of the scoring unit are implemented—one for each output of the calculator. Each unit accumulates the scores of the mass fragments that it receives; when the calculator has calculated all the masses around a hit, the scores from the individual units are accumulated. This total corresponds to a score for the hit. This score, paired with the hit location, is returned to the user as a set of ranked potential genes, which code the current unknown protein.

Results and Conclusions

As mentioned earlier, a new memory word is read into the device every cycle. Each word is 63 bits (21 bases) long and the search engine can operate at 92 MHz. This gives an effective search speed of 1.9 Gbases/sec. This unit resides on one of the four FPGAs on the TM3 and uses approximately 40% of the total look-up table (LUT) capacity.

The mass calculators and scoring units occupy the remaining three FPGAs with two frames on each chip. Each of the frame chips has 99% slice utilization (28K LUTs and 448K RAM bits). The congestion here restricts the routablilty of the circuit and limits the speed to 64 MHz. The Virtex 2000E have 43K LUTs and 614K RAM bits in total. On a larger device such as the Stratix S-80, with 79K LUTs and 7.4M RAM bits, the circuit will be far more routable and should be able to attain far greater speeds.

The device outlined here represents a prototype and must be integrated together with software that performs the initial sequence call for MS/MS data (e.g. Lutefisk (4) in order to provide input to the hardware. Output, in the form of a correlated list of addresses of hits in the database, their scores also must be integrated with software to present the information for further processing. Modules have been built that post-process the information to find canonical splice variant masses that can be further compared with the PIS mass list to identify splice overlap peptides and help solve the gene structure of detected proteins.

### EXAMPLE 2

This example describes a hardware system of the invention for sequencing proteins. The design of the system takes three primary inputs, namely:
1. A peptide query from the MS, which is a string of 10 amino acids or less,
2. A genome database,
3. A list of peptide masses detected by the MS.

The design produces a set of outputs for a given peptide query:
1. A set of gene locations, which can code the input peptide query
2. A set of scores for each gene location. The scores rank the genes based on the likelihood that they coded the protein in the sample.

The hardware identifies all locations in the genome that can code the peptide query and then translates these gene locations into their protein equivalents. It then compares the peptides in the translated proteins to the peptides detected by the MS and provides a ranking for each gene location based on how well it matches the masses detected by the MS. These gene locations can be translated to their protein sequence in a matter of a few milliseconds by using the genetic code or by using existing software packages (23) (24).

The design is divided into three major subunits:
1. A search engine that locates all possible coding strands for a peptide query.
2. A tryptic mass calculator that translates all matching genes and produces the masses of all the corresponding tryptic peptides from the translated gene.
3. A scoring unit that compares calculated peptides against those stored in the PIS of the MS and ranks the matching gene locations.

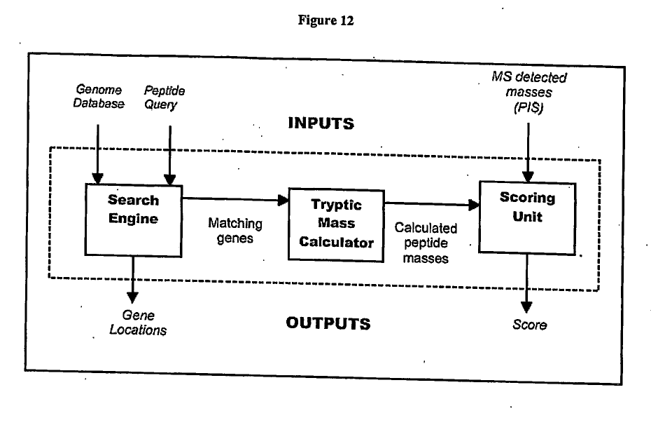

This architecture is depicted in FIG. 12. In the following sections the inputs are described and how they are encoded within the system is explained. Each of the units in FIG. 12 is described as the flow of data through the system is detailed.

Genome Database Coding and Compression

The genome database is one of the primary inputs to the system. To better understand the nature of operations performed on this database, a description of the data encoding schemes used to store this database is provided.

The genome database is stored as an ASCII file of bases, and is available for download from several different institutions. The ASCII representation uses 8 bits per character, which allows for 256 unique characters to be stored. However, since there are only 5 different characters (the four bases A, T, C, G and the wildcard N) in the genome database 98% of the storage space is wasted. This ASCII file is encoded using a different scheme that allows for better compression of the data. Each codon in the genome file is encoded using a 7-bit value that allows for 27=128 unique codons. Each codon consists of 3 characters and the characters themselves can be one of five values. Therefore there are 53=125 unique codons in the actual genome database. For example AAA=0000000, AAT=0000001, AAC=0000010 etc. This encoding uses 2.3 bits per base wasting only 2.3% of the storage space (125 of 128 possibilities used).

Since the genomes of most organisms are large (15 million to 3.3. billion characters), it is not practical to store the genome database directly on-chip. Instead the genome database in RAM is stored external to the FPGAs.

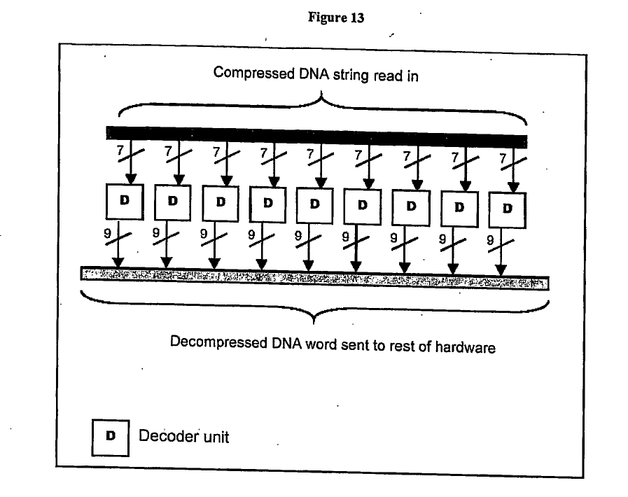

As the genome is read from external RAM into the device, it first passes through the decoder units illustrated in FIG. 13. Each decoder takes in a 7 bit “compressed” codon from memory and produces a 9 bit “uncompressed” codon using the original 3-bit encoding scheme. The decoders themselves are BlockRAM units that are configured as ROMs. They accept the compressed string as an address and produce an uncompressed bit-string as their output.

The uncompressed bit-string uses 3 bits per base that allows for eight possible characters, five of which are used (A=000, T=001, C=010, G=011 and N=100 for ambiguities). Thus a single codon is represented by a 9-bit value within the hardware as shown in FIG. 13. The rest of the hardware units described in the following sections also use the 3-bit encoding scheme described above.

Peptide Query

The output of the second MS in an MS/MS experiment is a peptide sequence (i.e. a string of amino acids). This must be converted to an equivalent DNA representation to be compared against a genome database. Consider for example the case when the MS outputs the peptide sequence “MAVR”. The goal of the algorithm is to locate all genes that can create this peptide.

Therefore each amino acid is translated into the codons that it could have originated from. The peptide query is a string of no more than 10 amino acids (including wildcards). This query size was chosen based on the average size of the sequencable portion of a tryptic peptide (approx. 10 amino acids) and the fact that a very short sequence of amino acids (often less than 7) can uniquely identify the protein it originated from (14).

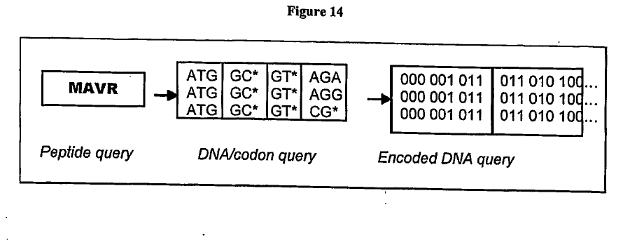

The wildcarding of searches is allowed by the inclusion of a wildcard character in the query. This also serves to compress the query, as some amino acids with multiple codons will not need each codon explicitly enumerated (for example the amino acid Alanine (A) in the query above is expressed as GC*). This reverse translation is done on the host PC when the peptide query is received from the MS. No more than three codons are needed to encode any amino acid when wildcards are employed. Thus each amino acid is reversed translated in the peptide to generate a codon, or DNA query that encapsulates all the possible coding strands for the peptide query as shown in FIG. 14. Each of these DNA/codon queries are then encoded using the 3-bit scheme described above.

Genetic sequences are stored as either original DNA strands or their complements, but never both, since this is redundant. In the 3-bit encoding scheme, no information is stored to indicate the type of strand. Therefore the complement of every strand in the database is considered to ensure that all possible coding patterns within an organism's genome are examined. For this purpose, the complement of the query is also generated. Thus the original peptide query is translated into six binary strings, three for the original DNA strand representation and three for its complement. The query, thus encoded, is submitted to the search engine, which locates all instances of the coding stands in the genome.

Search Engine

The primary objective of the search algorithm is to identify all possible locations in the genome from which a peptide may have originated. To accomplish this, the user provides a peptide query (inferred from the MS data), which is simply a string of amino acids. To compare these amino acids to a genome (DNA) database they must be reverse translated to codons. The search engine takes these strings of codons as input, and outputs all positions within the genome that match the strings.

The purpose of implementing the search in hardware is to maximize speed. This speed is governed by the frequency with which the memory containing the genome can be clocked through the search engine. The parameter MEM_WIDTH is defined to be the width of a memory word that is read into the search engine, i.e. the number of bits read into the system in every clock cycle. Thus the total number of clock cycles required to search through a genome in memory (with a size defined by SIZE_OF_GENOME) is given by:

$$\frac{SIZE\_OF \_GENOME}{MEM\_WIDTH}$$

Consequently the total time to search through the database is given by:

$$Total\_Search \_Time = \frac{SIZE\_OF \_GENOME}{MEM\_WIDTH} \times \frac{1}{System\_Frequency}$$

Note that the total search time must be less than 1 s for the search engine to be useful in the de-novo sequencing method. Furthermore, there may be other applications that require high-speed searches of the genome.

Search Engine Operation

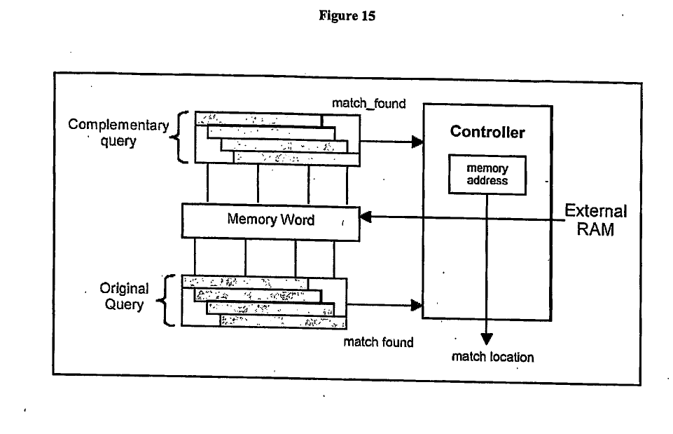

The search engine accepts queries, which consist of a set of DNA strings and their complements, and locates every position within the genome that matches any of these strings. The genome, which is stored in the RAM, is clocked in as a series of MEM_WIDTH-bit memory words. On every clock cycle the controller reads a new memory word into the system. This word is compared to the set of queries provided by the user. If a match is detected, the search engine controller returns the current memory address, which the user can then use to locate the coding gene. The VHDL (Very High Speed Integrated Circuit Hardware Description Language) description of the search engine controller is provided in Table 1 (control.vhd). A depiction of the architecture of this device is provided in FIG. 15.

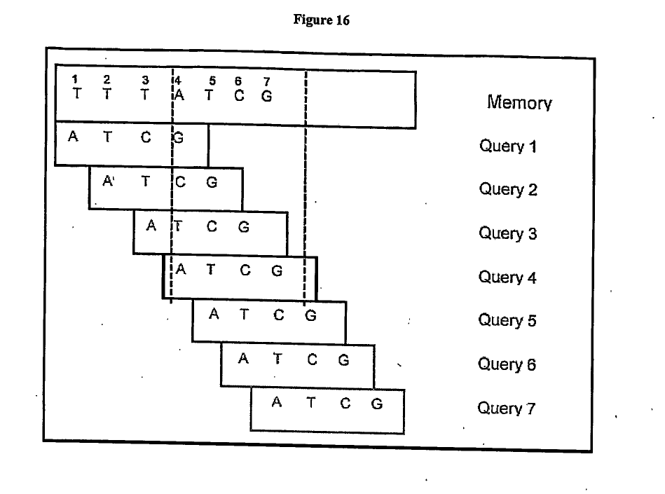

Once reset, the search engine controller enters initialization state in which the six DNA queries are read into the search engine. This is done in two clock cycles: one for the original DNA query, and one more for the complementary query. In the example in FIG. 16, a simplified view of the architecture is presented, in which a single DNA query is performed. Note that the complementary query shown in FIG. 15 is removed for simplicity, however the search operations performed on both strings are identical. The controller then moves into the comparison state in which memory words are continuously read into the search engine from external RAM. With a new word entering the engine in each cycle, every substring within the memory word must be compared to the query in a single cycle. To do this, multiple copies of the query are registered in hardware, and each one is simultaneously compared against the memory word. Note that as many copies of the query are needed as there are bases in the memory word. This is apparent in the architecture shown in FIG. 16 as each copy of the query is aligned with a successive base in the memory word.

Using the compression scheme of 7 bits per codon, the number of bases in a single memory word is parameterized as:
NUM_BASES_IN_MEMWORD=MEM_WIDTH×7/3

Each copy of the query is stored in a peptide unit, and if any peptide units signal a match (as query 4 in the example in FIG. 16), the controller exits the comparison state and returns the current memory address to the user, to be interpreted as a coding region for the query strand. The search engine then returns to the comparison state and the process continues until all the memory has been read.

It is apparent that the peptide units mentioned above are responsible for the core functionality of the search engine. To elucidate the details of the design, a description of the peptide unit follows.

Peptide Comparison Unit

The search process described above compares several identical copies of the query to a memory word to maximize throughput. Each query is stored in an individual peptide unit.

A peptide comparison unit takes two inputs:
(a) A set of query codons (corresponding to the amino acids in the query);
(b) A set of 10 codons from memory.

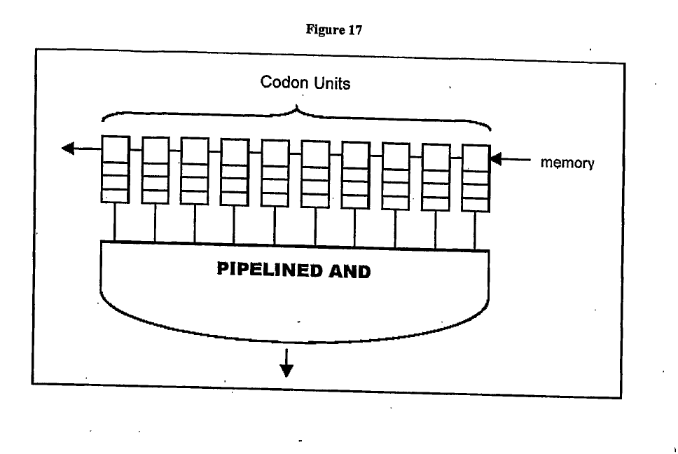

FIG. 17 represents the general architecture of a peptide comparison unit. The query codons are stored in a set of codon units. Each of these units then receives codons from the memory word, which are compared against the query codons. Each unit produces a single match output that signals whether the codon from memory matches any of the query codons. If all of these match signals are activated simultaneously, a string of codons from memory that matches a set of query codons has been found. The VHDL description that instantiates the peptide comparison unit is presented in Table 1 (protein.vhd)

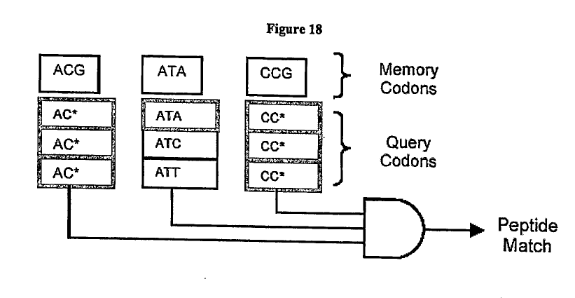

In FIG. 18 a simplified peptide comparison unit is depicted in operation. There are 3 sets of query codons, which are compared to the codons from memory. In FIG. 18 the matching codons are highlighted. If at least one codon from each set shows a match to memory, the query has been found in the genome, or equivalently, a coding strand for the peptide query has been found.

Thus each of the codon sets signals a pipelined logical AND unit, and if all sets indicate a match, the peptide unit signals a match. A wide AND operation (logical AND with many inputs) will incur significant delay if it is to be completed in a single cycle. To avoid this delay and ensure fast circuit operation, the match registers signals from the units, then AND them as a pipelined operation.

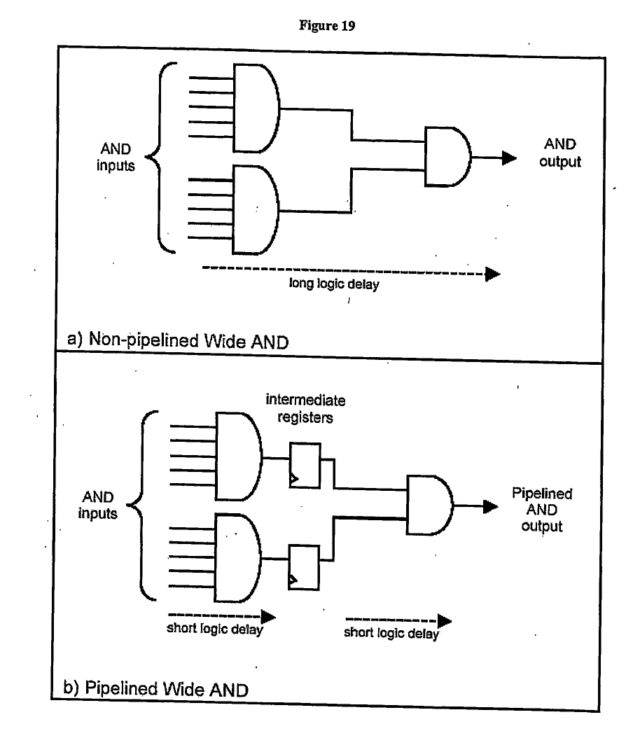

FIG. 19 contrasts a simple wide AND implementation with the pipelined version described above. In the non-pipelined unit, there is a comparatively long logic delay as the input pass through multiple gates to produce the output AND signal. If this delay is sufficiently high, it will constrain the maximum clock frequency of the circuit. In the pipelined implementation, the inputs are divided into two groups. Each of these groups is individually ANDed in a single clock cycle. The results of this operation are stored in intermediate registers and ANDed together in the next clock cycle. This technique reduces the delay through logic and allows faster circuit operation. Note that the output of the pipelined AND is delayed by an additional clock cycle, but this is usually acceptable as the clock frequencies are sufficiently high, and the penalty of an extra cycle is negligible.

FIG. 17 depicts the peptide unit as a set of codon units, as described above. It is the match signals from each of these codon units that are ANDed together to verify that all codons have detected a match in memory. These codon units are the building blocks upon which the search engine is built.

Codon Unit

The smallest fundamental unit of the search is the codon unit, which takes a set of three query codons and a single codon from memory as its input. It produces a match signal as its output. If any of the three query codons matches the memory codon, the match signal is activated. The set of three codons corresponds to the translation defined above. Any amino acid can be represented as set of three codons or less. Thus a codon unit essentially determines whether a codon from memory is capable of coding a query amino acid.

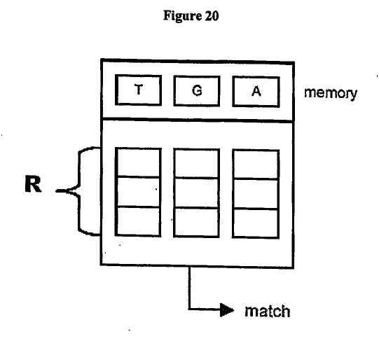

The operation of the codon unit is shown in FIG. 20. Assuming that the query amino acid is Arginine (R), it is translated to its equivalent codons AGA, AGG and CG*. This is done in software before the query is submitted to the search engine hardware. These three query codons are stored in the codon unit, and at every clock cycle, a new base from the genome in memory is read in and compared against the queries.

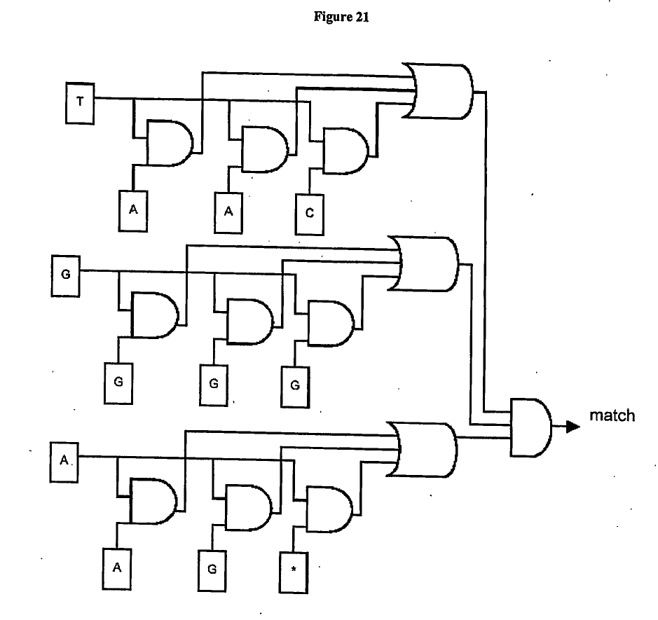

FIG. 21 illustrates a detailed view of the codon unit. The bases in the three query codons are divided by position, i.e. the first base in every query codon is ANDed with the first base for a codon from memory, the second query base is ANDed with the second memory base and so on. From FIG. 21, it is apparent that the codon unit only signals a match if each base from memory matches at least one query base in its corresponding position. The VHDL code that describes this architecture can be found in Table 1 (amino.vhd)

It is the match signal shown in FIG. 21 that is passed into the pipelined AND in the peptide comparison unit, and ultimately to the controller, which then detects a hit and returns the corresponding memory address to the user.

Interpreting Search Engine Outputs

The search engine identifies memory addresses that contain a section of DNA capable of synthesizing the query peptide. In a biological sense, this corresponds to identifying coding genes within the genome. FIG. 12 indicates that the gene at the hit location is then sent to the tryptic mass calculator for further processing.

However the stream of DNA from the genome database, which passes through the search engine, has no markers to indicate the start or end points of a gene. To overcome this lack of information, the average size of a gene is used to delineate the gene under consideration.

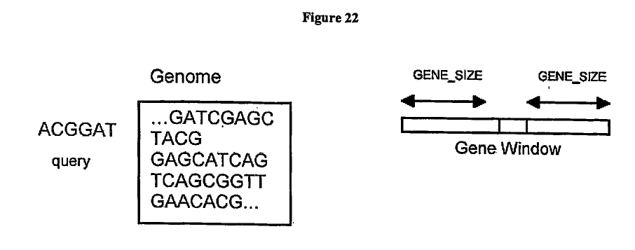

Defining the size of a gene as GENE_SIZE bases, a 2×GENE_SIZE window of bases surrounding the hit is sent to the calculator. This approach, as shown in FIG. 22, allows the consideration of one gene preceding the hit and one gene following it. In practice, this window is implemented as a GENE_SIZE sized shift register. The input data to this shift register is obtained from the output of the decoder blocks described herein. This data is in the uncompressed 3-bit form; therefore the depth of the shift register is GENE_SIZE×3 bits. Data from the decoder is continually passed into the gene window register, which acts like a delay element, as its outputs are delayed by GENE_SIZE (its depth) relative to its input. When the search engine detects a hit, the output of the gene window is sent to the tryptic mass calculator, which continues to read the gene window until it has processed 2×GENE_SIZE bases.

This technique ensures that the calculator processes a reasonable amount of genomic data on either side of the hit location. However, the fixed size of the gene window adds an inherent error to further operations, as most genes will be of a different size. Regardless, if a reasonable portion of the gene is processed, it will still be possible to identify many of the peptides from the translated protein.

Summary of Search Engine Design and Operation

The original peptide query is translated from amino acids to sets of codon. These codon strings are stored in the codon units that make up a peptide unit. Multiple identical copies of the peptide unit are instantiated to maximize the throughput of the search. The search engine progresses incrementally through the address space of the genome stored in RAM, looking for a match to the queries. If a match is found, the current memory address is sent to the user as a gene location that codes the peptide query. Genomic data surrounding the hit location is then sent to the Tryptic Mass Calculator as illustrated in FIG. 12.

Tryptic Mass Calculation

Overview

Referring to FIG. 12 the search engine locates genes matching the peptide query and sends the corresponding addresses to the user. It remains to translate all matching genes to their protein equivalent, digest these proteins to peptides and calculate the masses of the peptides. Peptide masses from each translated protein are then compared with the PIS list (Table 2) to determine which translated protein most closely matches the protein sample in the MS.

The tryptic mass calculator receives matching genes as its input, and performs the translation, digestion and calculation operations described above to provide the peptide masses as outputs. To do this the calculator unit must translate the matching genes from the search engine into amino acids and locate the tryptic cut-sites. To obtain tryptic peptide masses, the sum of masses of the amino acids from cut-site to cut-site is accumulated. These masses are then sent to the Scoring Units as illustrated in FIG. 12.

As an overview of the mass calculation process, an example of the steps involved is set out below.

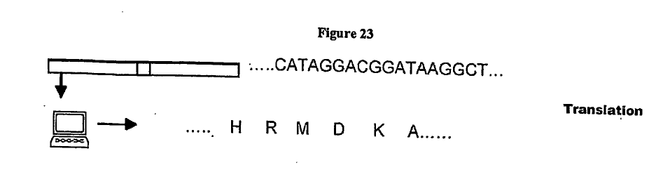

The DNA data from the gene window, i.e. the matching genes, are interpreted as a stream of codons, or equivalently, as an amino acid string. In effect, the gene is translated to its corresponding protein as shown in FIG. 23.

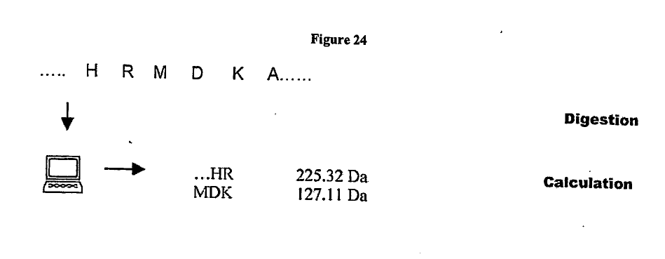

Once a protein is translated, its tryptic peptides must be compared to those detected by the MS. To identify the tryptic peptides and digest the protein, the calculator detects the tryptic cut-sites (Lysine (K) and Arginine (R) amino acids) and calculates the accumulated mass of all amino acids between these cut-sites as illustrated in FIG. 24.

Calculator Architecture

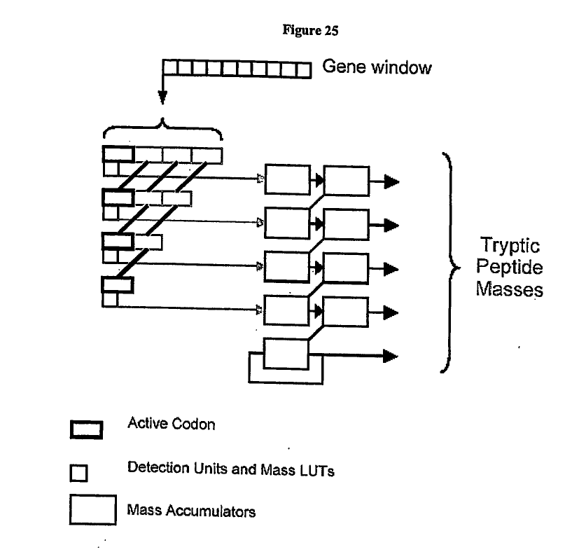

An architectural view of the calculator as depicted in FIG. 25 shows a pipelined design that performs the translation, digestion and peptide mass calculations described above.

At every clock cycle, the controller for the calculator reads a new set of NUM_BASES_IN_MEMWORD bases from the gene window into the calculator. The calculator operates on this data in codon-sized units. Note that each stage of the calculator in FIG. 25 has a single active codon attached to a detection unit and mass lookup table. The first stage of the calculator translates its first codon into the mass of its corresponding amino acid, which in turn is passed to a mass accumulator. In the next clock cycle the controller reads a new set of codons from the gene window into the calculator, and the remaining unprocessed codons from first stage are passed down. In the Tryptic Peptide Masses second calculator stage, the second codon is, processed in parallel with the first codon from the new set. The accumulator from the first stage passes its calculated mass to the second stage. Thus the mass of the first amino acid can be added to the mass of the second to calculate the mass of the peptide. If the detection units identify a tryptic cut-site (Arginine or Lysine amino acids not followed by Proline), digestion occurs and the accumulated peptide is output from the calculator. Each stage of the calculator operates in an identical manner by receiving a set of codons, performing calculations on only a single codon and buffering the rest. These remaining codons are passed to the next stage in the subsequent clock cycle and the process is repeated until the entire gene has been processed. The VHDL representation of the behaviour of the calculator is given in Table 1 (mod_calc.vhd).

The matching gene is passed as input to the calculator, NUM_BASES_IN_MEMWORD at a time to match the memory throughput. The calculator operates on these bases in codon-sized units; therefore NUM_BASES_IN_MEMWORD/3 codons (defined as NUM_CODONS) are clocked into the calculator in every cycle. To maintain this throughput, the calculator needs at least NUM_CODONS stages operating in parallel, as there could be at most NUM_CODONS peptides in a single memory word. However, if a peptide spans more than a single memory word, the accumulated mass from the first memory word will have to be saved until the tryptic cut-site is detected in one of the following memory words. Thus an extra pipeline stage is required to accumulate intra-word peptides, resulting in a total of NUM_CODONS+1 stages operating in parallel to ensure that the calculator can meet the memory throughput.

For every hit detected by the search engine, the calculator processes a full gene window of bases. Thus for every hit, the calculator operates for a total of GENE_SIZE/NUM_BASES_IN_MEMWORD corresponding to one cycle for every memory word in the genome. An additional NUM_CODONS+1 cycles are required to process the codons that will remain the pipeline of the calculator. The following sections provide a detailed description of the architecture of the hardware used to perform the mass calculations.

Mass Calculation

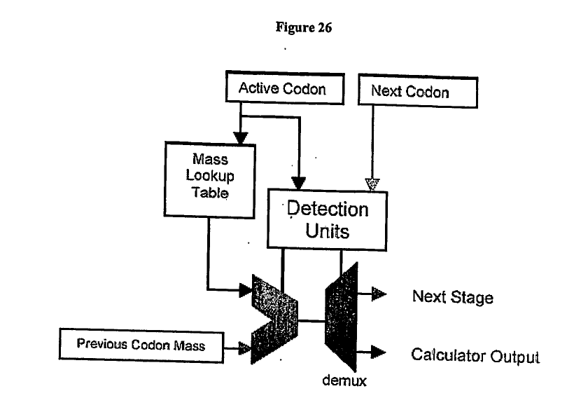

For a detailed account of the operations performed by the calculator, consider FIG. 26.

Each stage of the calculator only processes its active codon, which is fed into a lookup table of masses and a set of detection units. The mass lookup table reads the codon and produces the mass of the corresponding amino acid effectively translating the codon. The detection unit looks for tryptic cut-sites in the codon stream. If no cut-site is detected, the mass of the previous codon is added to the mass of the active codon. However, if a cut-site is detected, i.e. the end of a tryptic peptide is reached, the accumulated mass is sent to the calculator output instead. Thus the detection units and mass accumulators control the digestion and calculation operations of the calculator.

Mass LUTs and Detection Units

The mass LUTs are implemented as ROM tables which accept a 6-bit codon as input and provide a mass value, which is NUM_MASS_BITS bits wide, as output. A codon size of 6 bits implies that only 2 bits are used to represent each of the 3 bases in contrast to the 3-bit per base scheme described thus far. To explain this disparity, consider the binary representation of the codons. With only four real bases A, T, C and G, a two bit representation is sufficient to encapsulate all possibilities. The third bit is used to represent the wildcard character. Thus every mass is represented by two data bits and a single wildcard bit. As the mass lookup table is instantiated in BlockRAM, using a 9-bit input for every codon (3-bits per base) would require 2⁹⁼⁵¹² storage locations of NUM_MASS_BITS size in the BlockRAM. By using only the two data bits of a base, a codon can be represented in 6 bits. Such an implementation requires only 2-64 storage locations. The controller for the mass calculator uses the wildcard bit in combination with the wildcard detector to determine whether there is sufficient information to translate the codon into its amino acid mass.

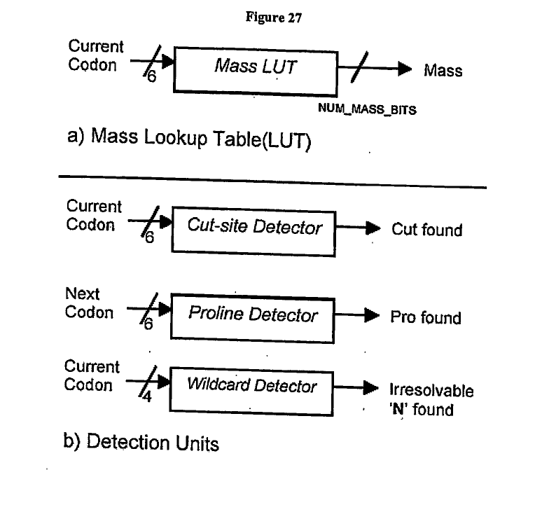

The cut-site detection unit looks for the presence of a Lysine (K) or Arginine (R) amino acid in the codon stream. Recall that trypsin cleaves the protein at these amino acids provided that they are not followed by Proline. Thus the Proline detection unit looks ahead to the next codon (see FIG. 27) to detect the presence of any codon that can synthesize the amino acid Proline. Both the cut-site and Proline detection units take a 6-bit codon as input and output a single bit indicating whether a cut-site or Proline codon was found in the input codon.

The wildcard detection unit looks for the presence of an irresolvable codon in the data from memory. The presence of a wild card or ‘N’ character in a codon does not automatically imply that the resultant amino acid cannot be resolved. In some of these cases, it is still possible to identify amino acid. The wildcard detection unit takes a 4-bit input (corresponding to the last two bases in a codon) and provides a 1-bit output, which is combined with the wildcard bits described above. The controller for the calculator uses this information to determine whether to save or discard the mass produced by a mass lookup table.

Complementary Strand Calculations

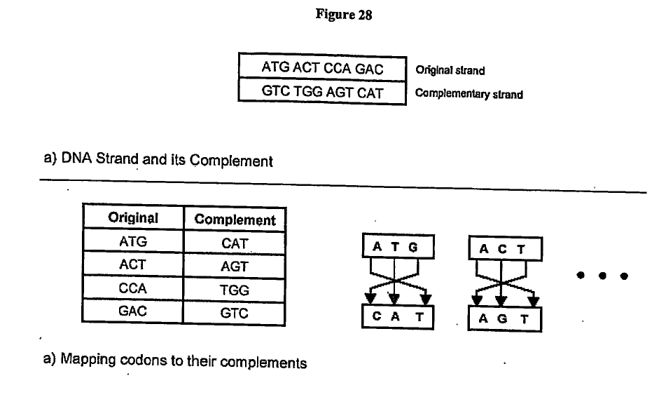

As with the search engine, the complementary DNA strand must be accounted for. The tryptic masses for both the strand stored in the genome, and its complement must be calculated. With the hardware above, the masses of tryptic peptides from the original strand can be calculated. For the complementary strand, a copy of this hardware is built which transposes and complements the codons. In FIG. 28 (a) an example string is shown alongside its reverse complement. Likewise, implementations of the cut-site, Proline and wildcard detection units for the complementary strand are instantiated within the calculator.

To obtain the reverse complement, the original strand is transposed and the bases are replaced with their complements. This corresponds to the reversed translation direction. However, the codons read from memory arrive in the order of the original strand and do not follow the transposed order depicted in FIG. 28 (a). Thus the codons are accumulated in the forward direction for the original strand (as read from memory), but backwards for the complementary strand.

This merely implies that, for the complementary strand, tryptic mass calculations will begin at the end of the protein. Mass accumulation is an associative process which is unaffected by the direction in which its input codons arrive.

Six Frame Mass Calculation

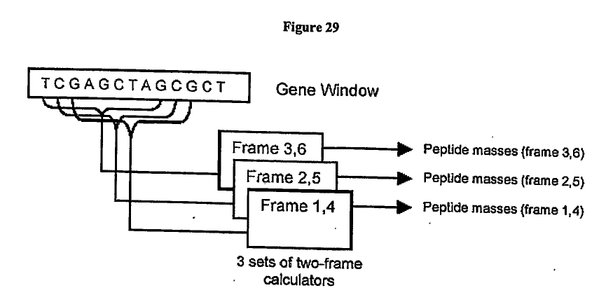

Each calculator unit computes the masses of one strand and its complement. This accounts for one frame and its complement. To account for the other two frames and their complements, two more calculator units are instantiated; each starts calculations at one base position ahead of its predecessor and operates identically to the structure described above. To implement this, output of the gene window shift register is read at different base locations by each of the three calculators as shown in FIG. 29.

Summary of Tryptic Mass Calculator Operations

The search engine identifies locations in the genome that can code the query peptide. The genes surrounding these locations are sent to the tryptic mass calculator to be translated into proteins and digested into tryptic peptides. The calculator then calculates the masses of these tryptic peptides. In the event that there are multiple matching genes, there is a list of tryptic peptide masses that correspond to each gene. These masses are compared with the peptide masses detected by the MS to uniquely identify the true coding gene.

Scoring Unit

Overview

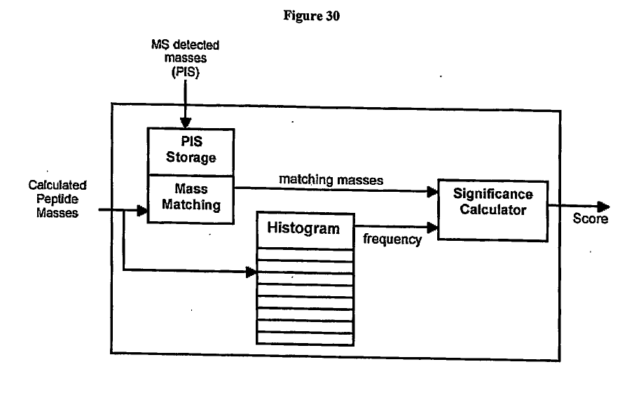

From FIG. 12 it can be seen that the calculator described herein produces the masses of tryptic peptides for all genes that coded the peptide query. These calculated masses are then compared with the masses detected by the MS to determine which gene actually codes the protein in the sample. FIG. 30 elaborates the representation of the scoring unit shown in FIG. 12. The VHDL description of this unit is in Table 1 (scorer.vhd)

The inputs to the scoring unit are the calculated tryptic masses and the PIS list from the MS. After comparing the two sets of masses, the unit produces a score indicating the quality match. Thus, the scoring unit serves to rank each hit (or gene window) in order of significance. Significance here is defined as the likelihood that a given gene window contains the gene that actually codes the protein in the input sample. The significance is computed using a histogram that records the frequency of occurrence of mass ranges. To compute this score, the hardware operates in three distinct states: True PIS storage, histogram construction and score calculation. In the first state the scoring unit merely saves the masses from the true PIS, which are primary inputs to the device. In the histogram construction state, peptide masses from the tryptic mass calculator are used to initialize the histogram. Once initialization is complete, the controller moves into the score calculation state in which it identifies matches between the calculated masses and those in the stored PIS. The matching masses are used in conjunction with the frequencies stored in the histogram to generate a score for the gene window.

The score consists of three major components: the product term, the maximum frequency and the number of matches. In the following sections, a description is provided of how the operations performed in the three states produce these three key components of the score.

True PIS Storage

Upon initialization, the masses detected by the MS (the true PIS) are sent as inputs to the scoring unit, which saves them in on-chip RAM. Later, as the calculator generates masses, each must be compared with the stored masses from the PIS. If they fail within a user-defined threshold of each other, a match is signaled.

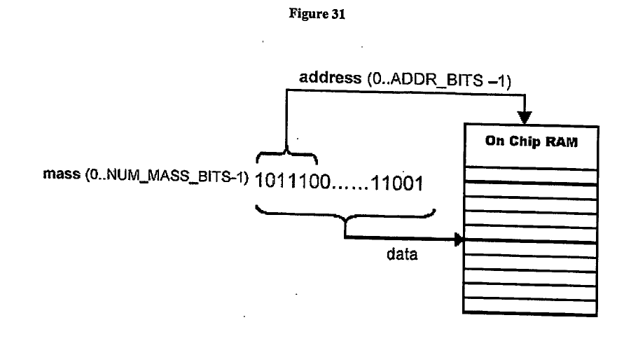

The first step in this process is to store the mass values from the MS in the on-chip RAM. The storage uses a data-associative indexing scheme similar to Content Addressable Memory (CAM). A subset of the most significant bits of the mass value is used to divide the masses into specific ranges as illustrated in FIG. 31.

In FIG. 31 a NUM_MASS_BITS sized mass value from the true PIS is sent to the on-chip RAM for storage. ADDR_BITS of the most significant bits from the mass value are used as an address into the on-chip RAM at which to store the mass. This storage method divides the masses into ranges; the range that a particular mass falls into is defined by its address. In the example in FIG. 31, the mass will be stored at address 46 (101110).

It is clearly possible for two different masses to be stored at the same address if ADDR_BITS of their most significant bits are identical. To avoid this situation, the design is constrained such that ADDR_BITS must be sufficiently large enough to ensure that data will not be overwritten. Upon device initialization, each of the PIS masses from the MS is stored in the on-chip RAM using this technique.

Histogram Construction

In the second state, the scoring unit initializes a histogram with NUM_BINS bins. As the mass calculator operates, its outputs are passed into the scoring unit. The histogram records the frequency of occurrence of peptides in different mass ranges. To this end, decoders are used to identify which range a given mass falls into and a set of counters is used to determine how many masses fall into a given range.

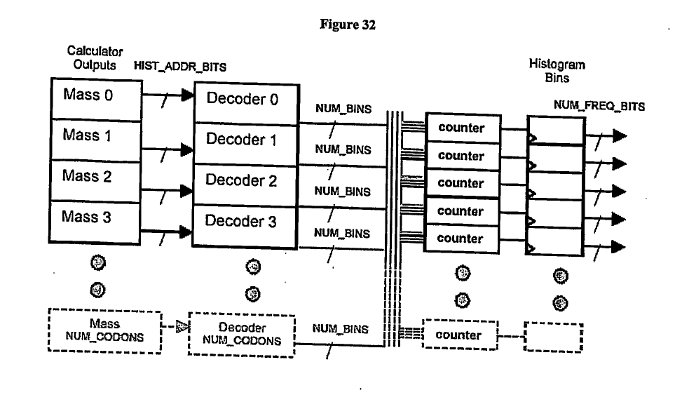

FIG. 32 illustrates how the decoders and counters described are used to update the histogram. Table 1 provides the VHDL description of controller that implements this process (mod frequency_table.vhd).

The bins in FIG. 32 are simply a set of NUM_BINS registers that are NUM_FREQ_BITS bits in width. Each register, or bin, represents a range of mass and contains the number of peptides in the current gene window that fall into this range. The counters at the inputs of these registers identify how many of the peptides from the calculator fall into a given range. The counter then updates the bin appropriately. The calculator is capable of producing NUM_CODONS+1 masses in a single cycle. Thus in every clock cycle, any bin in the histogram can be incremented by a maximum of NUM_CODONS+1 peptides.

As mentioned, binary decoders are used to determine the range into which a calculated mass falls. The decoder has log₂(NUM_BINS) inputs and NUM_BINS outputs. Each output signal of the decoder corresponds to one of the NUM_BINS bins. Therefore log₂(NUM_BINS) bits of the mass (defined as HIST_ADDR_BITS) are required to determine the range a given mass falls into. There are NUM_CODONS+1 decoders, each corresponding to single output of the calculator.

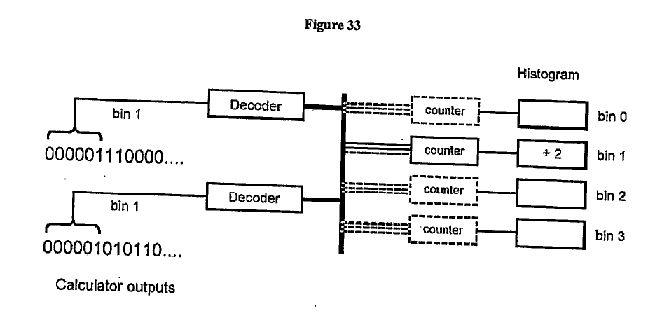

An example of a histogram update is presented in FIG. 33 for clarity. In this example two calculator outputs are shown. While both masses are different, HIST_ADDR_BITS of their most significant bits (6 bits in this example) are the same, thus both fall into the same bin (bin 1). Both decoders activate the output corresponding to bin 1, and the bin 1 counter correspondingly indicates that the histogram should increment the value in bin 1 by 2. Using this approach, the frequency of occurrence for each calculated peptide mass can be recorded. Once a full gene window has been processed, the bins are passed through a shift register, which identifies the mass range that occurs most frequently. The maximum frequency is one of the key components of the score and is returned to the user. The entire histogram update process occurs in parallel with the operation of the calculator, but an additional NUM_BINS cycles are required to identify the maximum frequency. The next phase uses this histogram to calculate the significance of the matching masses as shown in FIG. 30.

Score Calculation

Once the masses from the PIS have been stored and the histogram has been initialized, the score calculation process begins. This process consists of two operations that occur in parallel: mass matching and significance computation. The mass matching operation compares every calculated mass to the PIS values saved in the on-chip RAM to identify any matches. The significance computation uses these matching masses to determine the significance of the gene window at a hit location. The two remaining components of the final score, namely the number of matches and the product term are calculated by these operations. The following sections describe the architecture and operation of the hardware that implements these operations.

Mass Matching

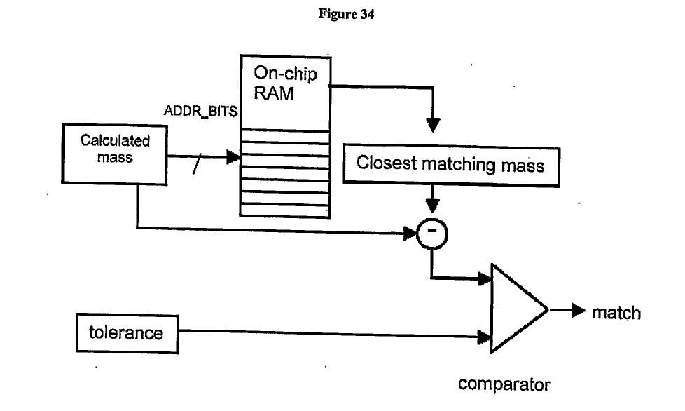

Once the histogram has been initialized, the masses from the tryptic peptide calculator are once again sent to the scoring unit. In this state however, the masses are not used to update the histogram. Instead, the calculated masses are compared with the true PIS masses that were stored earlier to identify any matches between the tryptic peptides in the current gene window and those detected by the MS. FIG. 34 represents the architecture implemented to perform the mass matching operations.

The goal of the mass matching hardware is to identify calculated masses that fall within a user defined threshold of a value in the true PIS. Given a tryptic peptide mass from the calculator, its closest corresponding mass is identified in the true PIS by once again using data associative techniques. To see how the closest matches are identified, recall the storage scheme used to save the true PIS.

The on-chip RAM, in which the true PIS masses are stored, is set into a read only mode and ADDR_BITS of the most significant bits of the masses from the calculator are used as addresses. Doing so retrieves the PIS mass that was stored at the same address, i.e. the retrieved PIS mass falls into the same range as the calculated mass.

The difference between the calculated mass and the stored PIS mass is then calculated. This difference is passed to a comparator along with a user-defined threshold. If the difference is less than or equal to the threshold, the comparator signals a match as illustrated in FIG. 34. The match signal is passed to the controller, which increments a counter to keep track of the total number of matches found in a window. This is one of the key components of the final score for the current gene window.

The matching masses identified here are used in the significance calculation step where the final component of the score, namely the product term, is computed. This process is detailed in the following section.

Significance Calculation for Matching Masses

In addition to the number of matches, the scoring algorithm ranks the matches by significance. FIG. 30 shows that the significance calculator receives frequency values from the histogram in addition to the matching mass values. The purpose of the significance calculator then, is to determine the ranges into which matching masses fall, and compute the product of the frequencies of these ranges. This corresponds to the product term.

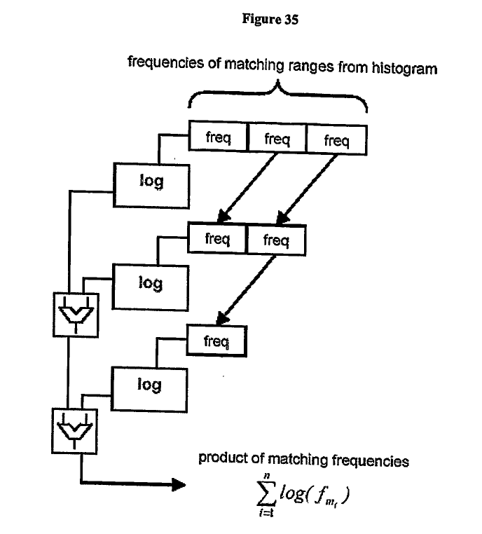

The peptide mass calculator can produce a maximum of NUM_CODONS+1 matching masses (i.e. every output of the calculator matches a mass value in the true PIS). To account for this event, the most significant HIST_ADDR_BITS bits of matching masses are used to identify the range the mass falls into. The frequency of this range is read from the appropriate bin of the histogram and placed in a pipeline as shown in FIG. 35. As with the tryptic mass calculator, the pipeline is used to ensure that the product of the frequencies of multiple matching masses can be computed per cycle to meet the throughput of the calculator. Each of the NUM_CODONS+1 stages of the pipeline processes a single frequency value per cycle. In the subsequent cycle, the unprocessed frequencies from every stage are passed to the following stage. However, the processing units depicted in FIG. 35 do not directly compute the product of the frequencies.

To calculate the product of the frequencies in the pipeline, the technique of logarithmic addition is employed as represented by the log and accumulator blocks in FIG. 35. This method relies on the fact that

$$log ( {\prod }_{i = 1}^{n} {f}_{{m}_{i}} ) = {\sum }_{i = 1}^{n} log ( {f}_{{m}_{i}} ) .$$

where fₘ corresponds to the frequency of a matching range and n is the total number of matches. Thus, instead of explicitly calculating the product of the frequencies, the sum of the logarithms of these values is taken. The actual product can be determined by taking the inverse of the logarithm of the accumulated value. This approach is primarily used to ensure that the product term can span a large range. The logarithm units are NUM_FREQ_BITS bits wide allowing for values between 0 to 2^{NUM}^{_{—}}^{FREQ}^{_{—}}^{BITS} to be represented. These values are calculated in hardware by lookup tables, which take a NUM_FREQ_BITS sized frequency value as input and produce log₁₀(frequency) as its output. Since the frequencies themselves are integer values from 0 to 2^{NUM}^{_{—}}^{FREQ}^{_{—}}^{BITS}, this simple scheme is sufficient to calculate the logarithms. The sum of these logarithms is computed by a set of accumulators to obtain the logarithm of the product term. This value is returned to the user, where the logarithm is inverted to obtain the final product term. This product term, along with the maximum frequency and the total number of matches between the hypothetical PIS and the MS detected values, is returned to the user to calculate the final score given by.

$$Score = \frac{1}{\frac{product\_term}{{( maximum\_frequency )}^{total\_number \_of \_matches}}}$$

A small product term indicates a match to an infrequent mass range, which corresponds to a high score. In practice, the actual score values produced by this formula vary in orders of magnitude i.e. high and low scores are typically several orders of magnitude apart. Therefore it is common for these scoring schemes to use 10 log(Score) as the final score value.

Six Frame Score Calculations

The calculators generate six frames of masses simultaneously. Each of these frames can be treated as an independent gene as each encodes a different set of tryptic peptides. Thus six corresponding scoring units, are instantiated in the hardware, each of which computes the score of an individual frame of the gene under consideration. Therefore each hit in the database is returned to the user with 6 sets of scoring information. Since only one of these six frames is the true coding region, the frame that generates the maximum final score for a given gene window is considered to be the true coding frame.

Design Summary

FIG. 12 illustrates an overview of the key subunits of the device.
1. A search engine that accepts a peptide query from the MS and locates all coding regions of the peptide in the genome.
2. A tryptic peptide mass calculator that translates and digests the genes around the located coding regions to produce the mass of the tryptic peptides that are contained in the proteins encoded by these genes.
3. A scoring unit that accepts the calculated tryptic peptide masses (the hypothetical PIS) and compares the calculated masses to the true PIS from the MS. The scoring unit assigns a score to each set of tryptic masses based on their significance. Each location identified by the search engine is associated with its score and returned to the user to determine the true coding region.

The design meets the speed requirements of current MS at a significantly lower cost than an equivalent algorithm implemented in software.

### EXAMPLE 3

Implementation Details & Results

Overview

A protein identification system described herein performs a reverse translated peptide query search through a Genome database. It locates all genes that can potentially code the query peptide and translates them into proteins. It then uses a variant of the MOWSE algorithm to compare the masses of these translated proteins to the masses in the PIS of a tandem mass spectrometer. This technique identifies and ranks potential coding regions for a protein or set of proteins in an MS sample. The coding regions can be sent to gene finding programs (24) (25) or homology search tools (19) to obtain the protein sequence.

Input Data

For this study MS data was used from the organism Saccharomyces cerevisiae, commonly known as baker's yeast. The yeast genome is an excellent model for the human genome since both are eukaryotes and thus share several similar proteins (21). The yeast genome (17) consists of 12070522 bases, which defines the parameter SIZE_OF_GENOME as 3.4 megabytes using the compression described herein. For comparison, the human genome is 918 megabytes.

Search Engine

In the search engine, the most crucial parameters are MEM_WIDTH and NUM_BASES_IN_MEMWORD, as they dictate the throughput of the system at a given operating frequency. The memory word read from the TM3A is 64 bits wide, but the compression scheme operates on multiples of 7 bits; therefore a MEM_WIDTH of 63 bits was used. The compression scheme uses 7 bits to encode a codon (or 3 bases) resulting in a NUM_BASES_IN_MEMWORD of 27 bases.

Gene Window

After passing through the search engine, the uncompressed memory word enters the gene window before it is sent to the calculator. The size of the organism's gene governs the size of the gene window upon which the calculator operates. Studies of the genes in yeast have shown the average gene size to be approximately 1450 bases (20). The gene window is thus implemented as 18-word 81-bit shift register (corresponding to a GENE_SIZE of 1458 bases). In contrast, the average gene size in human chromosome 7 is 70,000 bases with 10% of the genes as large as 500,000 bases. This expansion in size is due to more alternative splicing (55% of chromosome 7 genes are spliced as opposed to 4% in yeast) (28).

Mass Calculator

The bases from the gene window are read and translated by the calculator into peptide masses. Measurements on the dataset showed that tryptic peptides range in mass from 0 to 10 KDa a 20-bit mass value ((220=1048576) allows for masses between 0 and 10,485.76 Da. However for an additional level of precision, 5 more bits are used to further divide these masses into 0.0003125 Da ranges. Thus NUM_MASS_BITS is set to 25 bits.

Scoring Unit

The masses from the calculator are passed to the scoring unit, which ranks them in a similar manner to the MOWSE algorithm. MOWSE defines bins of 100 Da, which were approximated by setting NUM_BINS to 128 bins. In the mass range between 0 and 10,485.76 Da, this translates to bins of approximately 82 Da. The choice of 128 bins in turn defines HIST_ADDR_BITS as 7 bits, as 7 bits of the mass are needed to identify 127 bins.

For convenience, these design parameters are listed in Table 3.

TABLE 3
Design Parameters

| | Values | Values |
|---|---|---|
| Parameter | (Yeast) | (Human) |
| MEM_WIDTH | 63 bits | 63 |
| SIZE_OF_GENOME | 3.4 Megabytes | 917 Megabytes |
| NUM_CODONS | 9 cadons | 9 codons |
| GENE_SIZE | 1458 bases | 35000 bases |
| ADDR_BITS | 9 bits | 9 bits |
| NUM_MASS_BITS | 20 bits | 20 bits |
| NUM_BASES_IN_MEMWORD | 27 bases | 27 bases |
| HIST_ADDR_BITS | 7 bits | 7 bits |
| NUM_BINS | 128 bins | 128 bins |
| NUM_FREQ_BITS | 8 bits | 8 bits |

The parameter values in Table 3 are chosen for a design with sufficient resources to perform the scoring operations accurately. In the following section the implementation details of a device designed with these parameter values is presented.

Implementation Details

In this section, the particulars of the design implemented with the values in Table 3 are presented. Firstly, the functionality of the design when used with MS data is shown. In the subsequent sections, hardware and software platforms implementing the design at varying levels of performance are considered. Finally the costs of these systems are compared in an attempt to identify a practical solution.

Functionality

The following tests were performed to gauge the performance of the system with real MS data. The data used were obtained from the study performed in (33). The study utilized Liquid chromatography tandem mass spectrometry (LC-MS/MS) analysis using a Finnigan LCQ Deca ion trap mass spectrometer fitted with a Nanospray source. Protein identification was performed by the search engines Mascot (22), Sonar (35), Sequest (36) and PepSea (37). The input sample used in the experiment contains two well-characterized proteins from Saccharomyces cerevisiae (baker's yeast):
1. A Rab Escort Protein (REP) [ACCESSION: NP-015015]
2. A heat shock protein from the SSB2 variant of the HSP70 family [ACCESSION: NP 014190]
   Rab Escort Protein (REP)

The REP in the protein sample is from the MRS6 family of proteins created by the MRS6 gene, located in yeast chromosome 15. A full gene map is located on the Saccharomyces Genome Database (SGD) (18). Its coordinates in the database (i.e. the bases that the gene spans) are from 1025599 to 1026956. (located in Chromosome 15 (18)

Heat Shock Protein (HSP70)

The HSP70 family is coded by the SSB1 and SSB2 genes located on chromosomes 4 and 14 respectively. The sample contains the SSB2 subfamily variant coded by the gene in chromosome 14.

Each of these chromosomes codes a different subfamily of the HSP70 proteins but both have extremely similar sequences (BLAST (19) of the 2 sequences shows 551 out of 613 matching amino acids (89% identity)). A full gene map is located on the SGD. (located in Chromosome 4 (16), located in Chromosome 14 (17))

Its coordinates in the database are:
from 1427427 to 1429279. SSB1 variant (located in Chromosome 4)
from 9661724 to 9663575. SSB2 variant (located in Chromosome 14)

Table 4 lists the some of the peptides that were provided as queries to the search engine alongside the hit locations reported by the search engine.

TABLE 4
Query peptides and hit locations for HSP70 and REP

| | Query Sequences Hit | |
|---|---|---|
| Protein | (minimal query)² | Location(s) |
| REP | vpealqr | 1025938 |
| | (vpealq) | |
| | saavggptyk | 1026060 |
| | (saavg) | |
| HSP70 | nttvptik | 1428705 |
| | (nttvpt) | 9663002 |
| | llsdffdgk | 1428495 |
| | (llsdff) | 9662792 |
| | tgldisddar | 1428190 |
| | (tgldis) | 9662487 |
| | fedlnaalfk | 1428352 |
| | (fedlna) | 9662648 |

²The minimal query (in italics under the query) is the shortest peptide sequence that still identifies a unique coding region 90

The first important observation is that any query sequence greater than 5 amino acids in length always uniquely identifies a single coding region, eliminating the need for a scoring function. Note that the peptides from HSP70 are shown as originating from two hit locations. There are two variants of this family encoded by different genes, but having highly similar sequences. However the 11% difference in sequence guarantees that the set of tryptic peptides generated by both variants is not the same. The scoring system helps resolve the two hits and uniquely identify the protein in the sample.

TABLE 5
Score identifies subfamily variant in HSP70

| | | Location(s) | |
|---|---|---|---|
| Protein | Query Sequences Hit | (Gene) | Score |
| HSP70 | nttvptik | 1428705 (SSB1) | 62 |
| | | 9663002 (SSB2) | 89* |
| | llsdffdgk | 1428495 (SSB1) | 65 |
| | | 9662792 (SSB2) | 89* |
| | tgldisddar | 1428190 (SSB1) | 67 |
| | | 9662487 (SSB2) | 88* |
| | fedlnaalfk | 1428352 (SSB1) | 66 |
| | | 9662648 (SSB2) | 88* |

In Table 5, the HSP70 peptide queries are shown alongside their scores. In each case, the SSB2 encoding (indicated by the * next to the score) has a higher score, corresponding to the variant that is in the sample. Each of the queries shown above is 5 amino acids or greater in length. An average sequence detected from a tryptic peptide may be up to 10 amino acids in length, but shorter sequences are common. Further, it is possible that only a short sequence can be determined for a long tryptic peptide due to instrument limitations, sample contamination etc. These shorter peptide queries to the genome have lower resolution and will result in multiple matches. A few smaller peptides were considered to test the resolution of the scoring function. These peptides were also identified by the mass spectrometer, but are shorter than the average peptide length, thus they are likely to encounter multiple matches within the genome.

TABLE 6
Queries with multiple matches in REP

| | | Hit | |
|---|---|---|---|
| Protein | Query sequences | Location(s) | Score |
| REP | eyvpr | 1026605 | 79* |
| | | 6672335 | 76 |
| | | 2264445 | 66 |
| | ilfak | 1938133 | 96 |
| | | 1323971 | 90 |
| | | 5006575 | 89 |
| | | 6224783 | 84 |
| | | 1025581 | 72* |
| | | 5231459 | 71 |
| | | 9309092 | 70 |
| | | 3108258 | 61 |

In Table 6 the queries “iflak” and “eyvpr” both generate false positives as expected. The query “eyvpr” in Table 6 is ranked correctly, and the true coding location gets the highest score. However, the second query is ranked incorrectly, with the true hit being ranked fifth. Scoring functions are highly sensitive to the data that they operate on (25) and the MOWSE algorithm that was used was not intended for genome wide searches (6). In cases where the query sequence is short and cannot be resolved to a unique gene location, multiple peptide queries may be used to identify the true coding region. This approach relies on the assumption that multiple matches are random, which may not always be true. For example, Table 5 showed multiple matches due to the fact that the two hit locations coded proteins that were similar or homologues. These matches were clearly not random, however most of the cases with multiple matches are random and occur due to the volume of data contained in the genome (1).

To see how multiple sequences can resolve the random false positive matches, such as those in Table 6, the distribution of match locations were observed. Each match corresponds to a gene location that codes the query peptide. In non-homologous proteins it is unlikely that several proteins will share common peptide sequences. Peptide mass fingerprinting (PMF) techniques make use of this fact to use a few peptides to discriminate between tens of thousands of proteins in protein databases.

Any short peptide query will match the true gene location and may produce several false positives. Thus if several peptide queries are used, the matches will be clustered together (within the true coding gene) while the false positives will be randomly distributed throughout the genome.

This can easily be seen in the data in Table 6. The two true matches are only 1024 bases apart, which is within the size of a single gene. The next closest match occurs between the hit at 1026605 and 1323971, but these locations are 297366 bases apart. It is thus easy to identify the true hits as they are clustered together.

TABLE 7
Closest Distances between Match Locations

| “ilfak” hit | Closest Match in | Distance to closest |
|---|---|---|
| locations | “eyvpr” | match |
| 1025581 | 1026605 | 1024 |
| 1323971 | 1026605 | 297366 |
| 1938133 | 2264445 | 326312 |
| 3108258 | 2264445 | 843813 |
| 5006575 | 6672335 | 1665760 |
| 5231459 | 6672335 | 1440876 |
| 6224783 | 6672335 | 447552 |
| 9309092 | 6672335 | 2636757 |

Table 7 shows the distance between the closest matches using the two peptide queries from Table 6. Using this information, it was deduced that matches that are close to each other indicate the presence of peptides being coded by the same gene, which in turn corresponds to the true hit location. Thus, the inverse of the difference between match locations is used to identify the true coding gene.

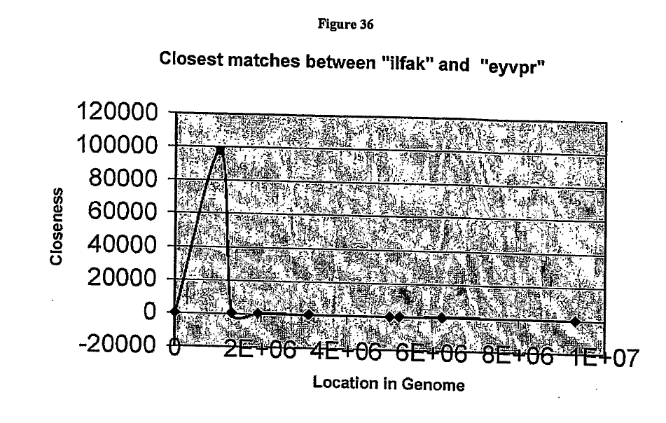

In FIG. 36 a scaled representation of the distance between the two queries is presented. The inverse of the distance between matches—which is defined as “closeness”—is presented across all bases in the genome in FIG. 36. The closeness value is scaled by a factor of 1×10⁷ for better visualization.

In FIG. 36 the true hit can be clearly distinguished from the other matches. Thus by using two peptides hits can be identified that cluster around a single gene and thereby discriminate a coding gene from random matches.

The short query peptides in Table 6 are natural, i.e., the peptides occur naturally via trypsin digestion. However, similar cases arise if the quality of the sample is poor and only a few amino acids can be sequenced. In these cases, the MS may only be able to resolve a short length of full tryptic peptide, forcing the MS operator to search the database with a shorter query.

To replicate the effect of these low quality samples searches were carried out using queries that are smaller than the minimal query. In effect, substrings of the queries in Table 4 were used to simulate the behaviour of “dirty” samples.

In the following example the two queries “saavggptyk” and “eyvpr” from Table 4 and Table 6 respectively are considered. To simulate low-quality sequences, the substrings “saav” and “eyvp” of these peptide sequences were used. However, the true hits are ranked 65th of 128 hits and 13th out of 48 hits for the queries “saav” and “eyvp” respectively. It is clear that the MOWSE scoring algorithm cannot distinguish the true coding locations from false positives. However, using the technique summarized in Table 7, the distance between hits can be examined. The 5 closest matches are presented in Table 8.

TABLE 8
Distance Between Hits in “eyvp” and “saav”

| “saav” Hit | Closest Match | Distance to |
|---|---|---|
| Locations | in “eyvp” | Closest Match |
| 1026060 | 1026605 | 545 |
| 7486943 | 7488841 | 1898 |
| 8964661 | 8965326 | 2305 |
| 10170118 | 10165117 | 5001 |
| 9383697 | 9378467 | 5230 |

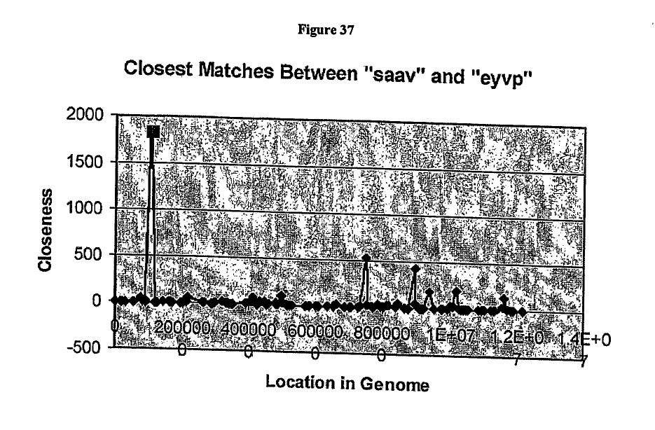

As before, the inverse of the distance to the closest match—the closeness—between hits produces a map of the genome in which the true coding gene is easily identifiable (FIG. 37). The true hit can easily be distinguished from 127 false positives, even when the query is only four amino acids long.

The results show that in many cases, the true coding region can be easily identified by using multiple queries. With a query of five amino acids, the true coding location was always correctly identified using two peptide queries to the database. When using a query length of four amino acids, the number of hits per query increases. With more hits, more queries are required to accurately identify the true coding region. Using two queries of length four identified the true hit in eight of 12 searches. Of the four erroneous cases, the true hit location is ranked 2nd in three of these and 3rd in the remaining case. In each of these cases, the distance between hits can be calculated in a few milliseconds, without significant impact on the speed of the search and score process.

Design Implementation on the TM3A

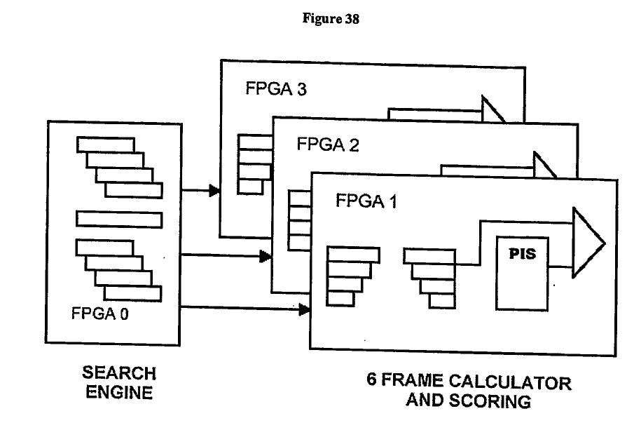

The TM3A described herein, was the primary implementation platform for the design. Considering the architecture of the TM3A, the device was partitioned across four FPGAs. The design is partitioned as shown in FIG. 38 and as follows:
FPGA 0: Search Engine and Gene Window
FPGA 1: Mass Calculator and Scoring Units (for Frames 1 and 4)
FPGA 2: Mass Calculator and Scoring Units (for Frames 2 and 5)
FPGA 3: Mass Calculator and Scoring Units (for Frames 3 and 6)

FPGAs 1, 2 and 3 have identical units implemented on them. The distinction lies in the data that they receive from the gene window. FPGA1 receives the data from the gene window directly, and produces the scores from Frame 1 and its complement (Frame 4). FPGA2 and FPGA3 receive the data from the gene window shifted by 1 base and 2 bases respectively, and correspondingly produce the scores of Frames 2 and 3 and their complements. Using this structure, the individual FPGAs can be classified by the units they implement. Therefore the design will be described in terms of search engine FPGAs and calculator and scoring unit FPGAs.

Compiling the design with the parameter values described in the previous section resulted in an implementation that did not fit on the TM3A due to insufficient resources. The 25-bit mass and 128-bin histogram force the calculator and scoring units to occupy more area than is available on a Xilinx Virtex 2000E FPGA. In combination, these units occupy 44338 LUTs and flip-flops, but Table 9 shows that the Virtex 2000 E chips on the TM3A only have 38,400 LUTs and flip-flops.

TABLE 9
FPGA resource comparison

| | Number of LUTs and | Block RAM | |
|---|---|---|---|
| FPGA | FFs | (bits) | User IO pins |
| Virtex 2000E | 38,400 | 655,360 | 804 |
| Virtex II 8000 | 93,184 | 3,024,000 | 1,108 |
| Stratix EP1-S20 | 18,460 | 1,669,248 | 586 |
| Stratix EP1-S40 | 41,250 | 3,423,744 | 822 |
| Stratix EP1-S80 | 79,040 | 7,427,520 | 1,238 |

In an attempt to fit the device on the TM3A, the design was modified to use 18-bit masses with a 64-bin histogram thus reducing the area occupied by the calculator and scoring units. This modification enabled the units to fit on the TM3A, and the speed and area results for the individual FPGAs are presented below.

TABLE 10
Total Resources and Speed for Search Engine on Virtex 2000 E

| | | | | Operating | Search Time |
|---|---|---|---|---|---|
| Design | | | Memory | Frequency | through Human |
| Platform | LUTs | FFs | (bits) | (MHz) | Genome(s) |
| TM3A - Virtex | 8,622 | 1,858 | 8,786 | 89 | 1.4 |
| 2000E | | | | | |

TABLE 11
Total Resources and Speed for Combined 2-Frame Calculator
and Scoring Units on Virtex 2000E

| | | | | | Processing |
|---|---|---|---|---|---|
| | | | | Operating | Time |
| Design | | | Memory | Frequency | for Human |
| Platform | LUTs | FFs | (bits) | (MHz) | Genome(s) |
| TM3A - Virtex | 27,925 | 12,475 | 34,816 | 58 | 2.1 |
| 2000E | | | | | |

The searching and scoring times shown are for the human genome, and not yeast. The ultimate goal of these sequencing experiments is to identify human proteins; the search times presented in Table 11 are more relevant when evaluating the practicality of the tool in useful biological experiments. The functionality of the device is not dependent upon the organism under consideration; indeed the only parameter affected in the value of SIZE_OF_GENOME, which is set to 918 megabytes (approximately 1 GB) when using the human genome.

From the tables above, it is apparent that the calculator and scoring units limit, and thus define, the system speed. Table 11 shows that it takes 2.1 seconds to identify and score all gene locations that match a single peptide query. This speed however is not achievable on the TM3A due to the limited speed of the SRAM. The operating frequencies in Table 10 and Table 11 apply only to the FPGA under consideration and are independent of memory speeds. The SRAM on the TM3A operates at a maximum frequency of 50 MHz making it the system bottleneck. Taking the memory speed into account, the operating frequency of the system is restricted to 50 MHz and the operating time is calculated for a single query to be 2.4 seconds.

In addition to the memory bottleneck, further problems arise as a result of the reduction in accuracy mentioned above. Using the less accurate 18-bit mass representation and coarser 64-bin histogram severely lower the performance of the scoring algorithm, thus the area and system speed presented above are not representative of a practical design. Note that this limitation only applies to the calculator and scoring units. The search engine fits on a Virtex 200013 FPGA and is not affected by the reduced parameters. Regardless, it is obvious that the TM3A, while a practical prototyping tool, is not adequately equipped to maximally implement this design.

To obtain realistic figures for area and speed, the design was recompiled with the parameters in Table 3 to target a set of modern FPGAs with more resources. These results are presented in the following section:

Design Implementation on Modern FPGAs

A new design implementing modern FPGAs and high-speed commercial memory is described below. The FPGAs under consideration are listed in Table 9. The newer FPGAs, namely the Xilinx Virtex II 8000 FPGA (31) and the Altera Stratix S40 and S80 FPGAs (32), all have more resources than the Virtex 200013 FPGAs on the TM3A. The Stratix S20 is included in Table 10 as it is the smallest FPGA upon which a search engine will fit. The speed and resource utilization tables are partitioned into individual FPGAs. The implementation of the search engine on each of the FPGAs is shown in Table 12. Correspondingly the implementation of the calculator and scoring units upon the Virtex II 8000 and Stratix S40 and S80 FPGAs is shown in Table 13. Due to the lack of resources on the Stratix S20, the calculator and scoring units do not fit on it.

Search Engine

TABLE 12
Total Resources and Speed for Search Engine using Current
Technology

| | | | | Operating | Search |
|---|---|---|---|---|---|
| | | | | Frequency | Time |
| FPGA | LUTs | Flip Flops | Memory Bits | (MHz) | (s) |
| Stratix S20 | 10,605 | 1,694 | 7,938 | 163 | 0.7 |
| Stratix S40 | 10,605 | 1,694 | 7,938 | 152 | 0.8 |
| Stratix S80 | 10,605 | 1,694 | 7,938 | 148 | 0.8 |

The reduced operating frequency on the larger devices in Table 12 can be attributed to the fact that the smaller devices have shorter wires, which have less capacitance, and are thus faster.

Two Frame Calculator and Scoring Unit

TABLE 13
Total Resourccs and Speed for Combined 2-Frame Calculator and Scoring
Units using Current Technology

| | | | | Operating | Search |
|---|---|---|---|---|---|
| | | | | Frequency | Time |
| FPGA | LUTs Flip | Flops | Memory Bits | (MHz) | (s) |
| Virtex II 8000 | 28,786 | 15,552 | 204,800 | 62 | 1.97 |
| Stratix S40 | 30,684 | 13,814 | 205,244 | 75 | 1.63 |
| Stratix S80 | 30,684 | 13,814 | 205,244 | 75 | 1.63 |

The difference between the number of flip flops and memory bits between the Virtex and Stratix FPGA can be attributed to the different synthesis and mapping tools used to implement the circuits. Various parts of the circuit are mapped to different structures (LUTs or BlockRAM) by the tools, which are tailored to find the best possible implementation of a circuit on a given device. The operating frequencies reported in the tables are independent of memory speeds and are based on a 63-bit memory word as indicated in Table 3. However, commercial DDR SDRAM was selected which operates in excess of 266 MHz (29), well above the system frequencies listed above, ensuring that memory will not be the bottleneck in the system.

The calculator and scoring units constitute the critical subsection of the design. From Table 13, a peptide query can be located and its coding regions ranked within 1.63 seconds, slightly over the 1 second requirement. By simply partitioning the genome into subsections and instantiating multiple copies of the hardware, the design can operate on each section simultaneously. Thus with two copies of the hardware, the entire search and score can be completed in 1.6312=0.82 seconds.

The data in Table 12 and Table 13 show that a hardware system capable of searching the genome at very high speeds can be designed using current FPGA technology in combination with existing commercial RAM. Capitalizing on the intrinsically parallel nature of the algorithm, hardware units at various levels of performance can be designed to meet a user's cost and performance requirements. However, the parallel nature of this algorithm lends itself to software implementation as easily as hardware. In the following section a software implementation of a similar algorithm is shown and the resources required to implement it are considered. This information will then be used to determine the most cost effective platform for this design.

Software

The software speeds and resources described here are taken from the study in (1). The scoring algorithm in the study is MASCOT, which is based on MOWSE. The operations in (1) were performed on a 600 MHZ Pentium III PC, resulting in search and score times of 3.5 minutes (210 s) per query. To scale these values to current processor speeds, a linear increase in speed was assumed if the algorithm is implemented on a modern processor. Based on this assumption, the software can complete the task in 52.5 seconds on a 2.4 GHz processor. This claim implies that the process will experience a speedup factor of 4 when run on a processor that is four times as fast. Such a scaling in speed is unlikely, as memory bandwidth does not scale with processor speed, but this assumption presents the ideal performance of this algorithm in software. A single modern processor currently cannot achieve the I-second search and score time.

As with the hardware, the algorithm is highly parallelizable and indeed MASCOT is a threaded program, designed to be implemented in a multiprocessor environment (22). To meet the 1-second operation time, the processing time scales were assumed perfectly with cluster size, i.e. to halve the time, the cluster size must be doubled. Table 14 shows the number of processors required to achieve performance that is comparable to the hardware.

TABLE 14
Processing Time for Computing Cluster

| Number of Processors | Processing time(s) |
|---|---|
| 1 | 52.5 |
| 32 | 1.6 |
| 64 | 0.8 |

Table 14 shows that a cluster of 64 processors can achieve the performance delivered by two copies of the hardware as described in the previous section. Thus both systems are capable of offering the same level of performance.

In the next section, the system is parameterized based on the resources required to achieve a user-defined level of performance. The required resources allow an estimation and comparison of the costs of the hardware and software systems to evaluate the most cost-effective solution.

System Cost and Resource Estimation

Cost of Hardware Platform for Full System

The most cost effective implementation for a system of the invention is achieved on a set of 4 FPGAs: one S20 for the search engine and three S40 FPGAs for the 6 frames of calculation and corresponding scoring units. Such a system requires sufficient RAM and a suitable PCB to act as a motherboard. The following is a selected design:
Each set of 4 FPGAs requires a 10.5″×14″-14 layer PCB as its motherboard.
Every search engine in the system has 2 GB of memory.

Multiple hardware units can be used to search subsections of the genome in parallel. Clearly a subsection of the genome will not require the storage space of the full genome. However, small memory modules are difficult to acquire commercially, and large memory modules can be purchased relatively inexpensively (29). Thus each hardware unit contains a full 2 GB of memory even though this is unnecessary for the design. A hardware system that takes under 1 second to search and score using a single peptide query, can be implemented for less than half of the acquisition cost of an equivalent software system.

The Stratix Power Calculator (34) is a tool that allows a designer to estimate the total power consumed by a design on a Stratix FPGA. Using the resource values from Table 12 and Table 13 the power consumed by the full hardware system is estimated as 7.6 W (1 W for a Stratix S20 containing search engine and 2.2 W for each of the three Stratix S40 containing the calculator and scoring units). The majority of the power is dissipated in the IO pins. All the FPGAs are running at 75 MHz and a 25% toggle rate is assumed for every flip flop and memory bit in the design. The custom hardware implementation consumes 200 times less power than general-purpose processor cluster. This reduction in total power consumption translates into a significantly lower operational cost over the lifetime of the cluster.

Cost of Hardware Platform for Standalone Search Engine

The search engine operating as an isolated unit does not require the same number of FPGAs or a PCB of the same complexity as the full system. Therefore the following design decisions are made for the standalone search engine:
A 10″×4″—8 layer PCB is required as the motherboard and can contain two FPGAs
Every search engine in the system has 2 GB of memory

Using these constraints, the Stratix S20 was found to be the most cost effective FPGA upon which to implement the search engine. The hardware searching system costs approximately 40 times less than a software platform of comparable performance.

The power savings are even more significant in the case of a clock speed of 162 MHz and a 25% toggle rate for every flip flop and memory bit, with the hardware providing over 2000 times the power to performance ratio of a software cluster. These results indicate that there are significant advantages to performing genomic searches in hardware.

Cost Comparison

This section summarizes the costs of the system, by dividing the solution into two broad categories, namely, low-performance and high performance. Here, low performance indicates search times in excess of a minute, which may be acceptable in many applications. However, the design must be able to identify and rank the coding locations for a peptide query in less than 1 second, thus demanding a high performance system.

For slower searches of the genome, i.e. search times in excess of 1 minute, software is a more cost effective solution than hardware. The software cost is based on the quoted price on a 2.4 GHz Dell Dimension Desktop (30). The cost of its hardware counterpart is based on the cost of a single hardware board capable of implementing the full system. It is possible to design a hardware system using cheaper, slower FPGAs but if real time performance is not required, a PC is likely a far more flexible solution with a greater capacity for reuse in other applications. Moreover, a PC at half the price of the hardware system is clearly a better choice. Therefore, at the low end of the performance spectrum, software is more practical vehicle for the searching and scoring process.

However, using the current cost and performance of the system as a measure of quality, hardware is clearly a better solution for an entity seeking the ability to search through genomes in real-time. At the high-performance end of the cost spectrum, hardware is more than three times as economical for equivalent level of performance. For a standalone search engine, hardware is more than 40 times as economical, making it an ideal platform for genomic studies.

The costs do not take power consumption into account. However, the performance to power ratio is far more favourable for hardware, than a cluster of general-purpose processors. Over the operational lifetime of the hardware platform, the power savings will likely translate to a substantial reduction in operational cost when compared with software.

The key resources that determine this cost of a hardware system are: the FPGAs, the RAM and the PCB. The FPGA (26), RAM (29) and PCB (27) costs are obtained from current vendor and manufacturer quotes. System designers in the future will likely have access to FPGAs with far more resources for which prices cannot be accurately predicted. As such the resources required for a given level of performance are defined. Knowledge of the required resources will allow selection of the most practical platform upon which to build the hardware.

In general, to design a system that meets a specific level of performance, the required resources can be estimated by the three elements listed above: FPGAs, RAM and PCBs. The total cost of the hardware is then given by the number of FPGAs (defined as NUM_FPGAs), the total amount of RAM (TOTAL_EXT_RAM) and the number of PCBs (NUM_PCBs). This cost is a function of the desired level of performance specified by the designer. The performance is specified by the time required to process an entire genome, thus the two variables that determine the hardware resources for the system are size_of genome (in GB) and search_time (in seconds). Thus the performance factor:

$$P = ⌈ \frac{size of genome}{search\_time} ⌉$$

The designer can use the desired value of P to determine the cost of the system in the future. This cost is given by:
COST(P)=(NUM_FPGAs(P)×FPGA_PRICE)+(TOTAL_EXT_RAM(P)×RAM_PRICE)+(NUM_PCBs(P)×PCB_PRICE)

An FPGA is classified in terms of its key components, namely the LUTs, flip-flops and memory and user 10 pins. Given these parameters, it is possible to determine the most cost-effective FPGA or set of FPGAs. The total number of LUTs and flip-flops in a given FPGA is defined as FPGA_LUTs_FFs, and the total on-chip RAM as FPGA_RAM, and the number of user IO pins as FPGA_IO_PINS. Using these parameters, a designer can determine the optimal FPGA for the device.

The following results are divided into two units: one to provide resource estimates for the full search and score system and the other for the search engine as an independent unit.

Resources Required for Full Search and Score System:

The values for each of these parameters depend on the performance factor P described above. A full implementation of the device from Table 11 requires 12,299 LUTs and flip-flops for the search engine and 3 LUTs and flip-flops for the calculator and scoring functions. Thus, with FPGA_LUTs_FFs=145313, a 1 GB genome can be processed in 1.6 seconds. To generalize this it can be stated that:
FPGA_LUTs_FFs=232500×P

Correspondingly, the device in Table 12 requires 7938 on-chip memory bits for the search engine and bits for the 3 calculators and the associated scoring functions. Thus 623670 on-chip memory bits are required to process the 1 GB genome in 1.6 seconds. This can be stated as:
FPGA_RAM=997872×P

The design requires a total of 1014 pins to process the genome as described. This enables the following definition:
FPGA_IO_PINS=1623×P

Using these three parameters, the value of NUM_FPGAs can be determined based on the most cost effective devices available at the time. To determine the optimal number of FPGAs, the cost and resources of a few large FPGAs can be compared with those on many smaller FPGAs. The most favourable solution implements the required resources at the minimum cost, thus defining the ideal value for NUM_FPGAs.

The next significant parameter is the amount of external RAM required. A single copy of a 1 GB genome can be searched in 1.6 seconds. As the level of parallelism increases and additional copies of the device are used to increase the system speed, multiple copies of the genome must be processed. This is generalized as:

$$TOTAL\_EXT \_RAM = \frac{1}{0.625} \times P$$

Using a design described herein as a reference, it is estimated that four FPGAs and the RAM can be connected on a single PCB without prohibitive complexity. This leads to the formula:

$$NUM\_PCBs = \frac{NUM\_FPGAs}{4}$$

The value of NUM_PCBs clearly hinges on an assumption of 4 FPGAs per board as defined in the design. The trend towards larger FPGAs implies that the design will eventually be able to fit on a single FPGA.

Each of these formulas is based on the design of the full search and score algorithm that operates on a 1 GB genome in 1.6 seconds. The formulas are intended to provide a sense of the required resources as the speed, and correspondingly the level of parallelism, within the system increase. If the required search time is less than 1.6 seconds, or the size of the genome is significantly less than 1 GB, the approximations provided here will be of limited value, as the formulas encapsulate the trend in resource requirements for increasing levels of parallelism.

Resources Required for Standalone Search Engine:

For the standalone search engine, the resource requirements can be defined as a function of search_time and size_of_genome to allow the user to estimate system costs in the future. The formulas given below are based on the data in Table 12 and Table 13 and assume a standalone search engine can search a 1 GB genome in 0.8 seconds.
FPGA_LUTs_FFs=9839×P
FPGA_RAM=6350×P
FPGA_IO_PINS=313×P

Once again, the actual value for NUM_FPGAs hinges on the technology available to the designer and can be determined based on the cost of available devices.

$$TOTAL\_EXT \_RAM = \frac{1}{1.25} \times P$$

When the design is constrained to two FPGAs per PCB the following formula results:

$$NUM\_PCBs = \frac{NUM\_FPGAs}{2}$$

The caveats from the first set of formulas apply equally well to the approximations above. The formulas convey the trends in resource usage based on the search of a 1 GB genome in 0.8 seconds.

The formulas above model the resources required for various levels of parallelization, which in turn correspond to different levels of performance. As stated the performance is dictated by the time taken to process a genome of a given size. Using the resources estimation models above the resources required to implement either the full search and score system described or the search engine as an independent unit can be estimated. These resources can then be used to determine the cost of the optimal solution based on the prices of available devices.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 2
Precursor Ion Scan (PIS) Masses
The following values (in Daltons)
were used to obtain the results in Chapter 4.

453.17
459.11
459.18
463.11
463.13
464.04
464.1
464.1
464.12
488.33
497.41
502.94
503.03
503.06
503.08
503.08
503.39
504.16
505.15
508.69
511.93
517.57
520.05
520.07
521
521
521.4
521.76
526.07
527.74
527.74
531.95
532.1
534.71
538.06
538.11
547
547.04
550.04
552.57
552.75
556.92
557.1
561.76
564.43
567.35
569.12
576.44
577.53
577.71
582.04
583.57
584.71
584.74
590.98
591.5
591.58
592.69
593.06
593.63
593.68
593.96
595.38
596.17
596.32
596.72
606.95
608.43
608.43
610.02
610.07
610.1
610.17
611.38
620.66
621.71
622.04
622.18
624.12
624.18
624.95
625.96
633.67
638.24
639.31
639.97
640.16
640.62
643.65
643.7
649.74
650.31
655.43
657.1
659.24
664.52
664.8
665.45
665.71
672.61
672.71
673.57
674.46
676.69
678.39
678.46
678.48
682.44
682.53
683.72
684.01
684.04
686.56
687.27
687.45
687.62
687.93
688.01
688.22
689.69
692.35
694.42
696.36
698.11
699.52
702.75
708.48
709.69
712.19
712.5
714.61
714.63
716.22
720.95
722.19
722.41
722.44
722.63
723.18
727.32
729.4
730.61
730.71
730.75
730.86
731.54
736.46
736.55
740.21
740.7
741.71
741.73
744.57
747.8
757.93
758
758.49
758.54
761.49
769.74
772.26
775.95
777.47
777.64
783.19
783.26
785.07
785.15
785.81
788.48
788.51
792.43
792.69
798.06
798.42
798.69
799.03
804.64
804.85
806.33
807.04
807.22
807.3
807.56
812.49
812.51
812.72
815.41
816.34
816.66
817.48
817.52
821.31
821.4
822.39
822.42
824.14
831.04
831.06
838.69
838.73
839.54
842.54
842.56
847.76
847.96
851.35
855.52
865.72
865.72
865.74
865.79
868.01
871.59
872.8
873.47
873.52
875.44
875.48
882.45
882.46
885.45
886.06
886.31
891.5
891.5
901.45
905.75
905.75
907.44
917.78
919.41
922.41
922.47
924.92
929.41
937.26
944.51
945.75
948.16
948.23
952.69
962.37
962.4
962.49
965.22
965.33
965.41
965.42
966.55
976.07
986.1
990.52
1000.39
1000.48
1002.49
1004.77
1006.65
1006.7
1008.59
1011.04
1011.19
1013.69
1014.12
1014.47
1020.78
1021.7
1022.19
1022.95
1023.29
1024.31
1028.24
1032.25
1032.42
1035.94
1041.68
1041.69
1050.67
1050.67
1053.72
1053.88
1056.25
1056.64
1057.6
1062.68
1062.7
1062.9
1064.19
1065.45
1067.52
1068.56
1076.29
1077.61
1078.48
1078.51
1080.77
1080.97
1082.65
1082.67
1084.17
1084.2
1084.63
1088.4
1088.95
1090.61
1090.67
1091.02
1093
1097.96
1098.55
1101.42
1102.21
1112.46
1112.48
1115.52
1117.49
1119.22
1125.57
1126.47
1131.73
1131.76
1137.67
1147.4
1156.42
1157.51
1158.55
1167.59
1167.64
1169.59
1181.69
1181.78
1185.82
1185.83
1190.67
1191.48
1199.77
1205.45
1206.73
1209.96
1210.01
1210.03
1210.23
1210.28
1213.44
1214.66
1218.78
1218.79
1220.15
1220.98
1224.15
1224.6
1226.14
1226.49
1232.64
1237.18
1242.72
1242.77
1247.38
1247.86
1256.67
1260.67
1260.7
1265.78
1270.63
1270.63
1277.5
1278.67
1278.68
1284.53
1296.56
1314.57
1316.68
1324.98
1343.66
1357.45
1359.44
1369.56
1371.64
1375.77
1375.82
1377.61
1383.37
1384.63
1386.25
1392.41
1409.32
1419.51
1424.64

6. Histogram Architecture (mod_frequency_table.vhd)

```
library ieee;
use ieee.std_logic_1164.all;
use ieee.std_logic_arith.all;
use ieee.std_logic_unsigned.all;
entity mod_frequency_table is
    generic( num_stages : integer := 10;
      num_freq_bits : integer := 8;
      size : integer := 8*8 ;
      shift : integer := 8;
      num_bins: integer := 128 );
port (
  clk : in std_logic;
  rst : in std_logic;
  enb : in std_logic;
  evaluate_mass : in std_logic;
  max_freq : in std_logic_vector(0 to 5);
  save_freq : in std_logic;
  low_freq_peptides : out std_logic_vector(0 to num_stages−1);
  mass_valid : in std_logic_vector(0 to num_stages−1 );
  matching_stages : in std_logic_vector(0 to num_stages−1);
  hist_max_freq : out std_logic_vector(0 to num_freq_bits−1);
  Pi_f : out std_logic_vector(0 to num_freq_bits−1);
  mass_ranges : in std_logic_vector(0 to (num_stages*7)−1)
);
end mod_frequency_table;
architecture mod_stats of mod_frequency_table is
-- decoder to decide which range is being incremented
component bin_decoder
  port (
  address: IN std_logic_VECTOR(6 downto 0);
  clock: IN std_logic;
  q: OUT std_logic_VECTOR(127 downto 0);
  clken: IN std_logic);
end component;
-------------------------------------------------------
-- ROMs to help count the total number of matches
component count_rom
  port (
  address: IN std_logic_VECTOR(7 downto 0);
  clock: IN std_logic;
  enable: IN std_logic;
  q: OUT std_logic_VECTOR(3 downto 0));
end component;
-------------------------------------------------------
-- check to see if any of the frequency bins meet low thresh
component or_34
Port (
          clk : in std_logic;
        or_in : in std_logic_vector(127 downto 0);
  or_out : out std_logic);
end component;
-------------------------------------------------------
-- log conversion LUTs
component logtable
  port (
  A: IN std_logic_VECTOR(5 downto 0);
  CLK: IN std_logic;
  QSPO_CE: IN std_logic;
  QSPO: OUT std_logic_VECTOR(7 downto 0));
end component;
-------------------------------------------------------
  type freqStates is (reset,update_stats,locate_max_freq,rank_masses);
  signal currState : freqStates;
  signal nextState : freqStates;
  signal full_max_freq : std_logic_vector(0 to num_freq_bits−1);
  signal element_counter : std_logic_vector(6 downto 0);
  signal hist_max_freq_reg : std_logic_vector(0 to num_freq_bits−1);
  signal frequency : std_logic_vector(0 to (num_bins * num_freq_bits)−1);
  signal saved_freq : std_logic_vector(0 to (num_bins * num_freq_bits)−1);
  signal saved_frequency_table : std_logic_vector(0 to (num_bins * num_freq_bits)−1);
  signal increment_range : std_logic vector(0 to (128*num_stages)−1);
  signal rev_increment_range : std_logic_vector((128*num_stages)−1 downto 0 );
  signal increment_amount : std_logic_vector(0 to (num_bins*4)−1);
  signal addr : std_logic_vector(0 to (num_bins*8)−1);
  signal bin_incr : std_logic;
  signal flagged_ranges : std_logic_vector(0 to (num_bins*num_stages)−1);
  signal freq_table_copies : std_logic_vector(0 to (num_freq_bits*num_bins*num_stages)−1);
  signal low_freq_range : std_logic_vector(0 to num_bins−1);
  signal pipe_mass_valid : std_logic_vector(0 to num_stages−1);
  signal matching_mass : std_logic_vector(0 to num_stages−1);
  signal frequency_pipeline : std_logic_vector(0 to (num_freq_bits*num_stages)−1);
  signal log_accum : std_logic;
  signal logadder_pipe : std_logic_vector(0 to (num_freq_bits* (((num_stages*num_stages)+num_stages)/2) )−
1);
  signal log_val_stages : std_logic_vector(0 to (num_stages*num_freq_bits)−1 );
  signal log_val_accum : std_logic_vector(0 to (num_stages*num_freq_bits)−1);
  signal temp_test :std_logic_vector(0 to (num_stages * num_freq_bits)−1 );
begin
  rev_increment_range <= increment_range ;
  full_max_freq <= “00” & max_freq;
log_convert : for i in 0 to num_stages−1 generate
convert_freq : logtable port map(
  A => logadder_pipe( (( size+(size*(i−1) − shift*(((i−1)*(i−1) + (i−1))/2)) ) + 2) to (( size+(size*(i−1) −
shift*(((i−1)*(i−1) + (i−1))/2)) ) + 7) ),
  CLK => clk,
  QSPO_CE => evaluate_mass,
  QSPO => log_val_stages( i*num_freq_bits to (i*num_freq_bits) + (num_freq_bits−1) )
  );
end generate log_convert;
range_selectors : for i in 0 to num_stages−1 generate
                range_decoder : bin_decoder port map(
                  address=> mass_ranges( 7*i
to (7*i + 6) ),
                  clock => clk,
                  clken => mass_valid(i),
                  q => increment_range(128*i
(128*i)+127)
                );
              end generate range_selectors;
incrementors: for i in 0 to num_bins−1 generate
              range_increment_value: count_rom port map (
                address => addr(i*8 to (i*8)+7),
                clock => clk,
                enable => bin_incr,
                q => increment_amount(i*4 to (i*4)+3)
              );
            end generate incrementors;
good_ranges : for i in 0 to num_stages−1 generate
          check_mass_range: or_34 port map (
            clk => clk,
            or_in => flagged_ranges(i*128 to (i*128)+127 ),
      or_out =>low_freq_peptides((num_stages−1)−i)
    );
            end generate good_ranges;
  process(currState,evaluate_mass,save_freq)
  begin
      bin_incr <= ‘0’;
    case currState is
      when reset =>
        nextState <= update_stats;
      when update_stats =>
        bin_incr <= ‘1’;
        if save_freq = ‘1’ then
          nextState <= locate_max_freq;
        else
          nextState <= update_stats;
        end if;
      when locate_max_freq =>
        if element_counter = “1111111” then
          nextState <= rank_masses;
        else
          nextState <= locate_max_freq;
        end if;
      when rank_masses =>
        if evaluate_mass = ‘0’ then
          nextState <= update_stats;
        else
          nextState <= rank_masses;
        end if;
      when others =>
    end case;
  end process;
  process(enb,clk)
  begin
    if rst = ‘1’ then
      currState <= reset;
    elsif rising_edge(clk) then
    if (enb = ‘1’) then
      currState <= nextState;
      pipe_mass_valid <= mass_valid;
      matching_mass <= matching_stages;
    logadder_pipe <= (others => ‘0’);
      logadder_pipe(64 to 119) <= logadder_pipe(8 to 63);
      logadder_pipe(120 to 167) <= logadder_pipe(72 to 119);
      logadder_pipe(168 to 207) <= logadder_pipe(128 to 167);
      logadder_pipe(208 to 239) <= logadder_pipe(176 to 207);
      logadder_pipe(240 to 263) <= logadder_pipe(216 to 239);
      logadder_pipe(264 to 279) <= logadder_pipe(248 to 263);
      logadder_pipe(280 to 287) <= logadder_pipe(272 to 279);
      for i in 0 to num_bins−1 loop
        addr(i*8 to (i*8)+7) <= rev_increment_range(i) & rev_increment_range(i+128)
& rev_increment_range(i+(2*128)) & rev_increment_range(i+(3*128)) & rev_increment_range(i+(4*128)) &
rev_increment_range(i+(5*128)) & rev_increment_range(i+(6*128)) & rev_increment_range(i+(7*128));
      end loop;
      for i in 1 to num_stages−2 loop
```

| | |
|---|---|
| log_val_accum(i*num_freq_bits | to |
| (i*num_freq_bits)+(num_freq_bits−1)) <= log_val_accum((i−1)*num_freq_bits to | ((i− |

```
1)*num_freq_bits)+(num_freq_bits−1)) + log_val_stages( (i+1)*num_freq_bits) to ((i+1)*num_freq_bits) +
(num_freq_bits−1));
      end loop;
          log_val_accum( (num_stages−1)*num_freq_bits to ( (num_stages−
1)*num_freq_bits)+(num_freq_bits−1)) <= log_val_accum((num_satges−2)*num_freq_bits to ((num_stages−
2)*num_freq_bits)+(num_freq_bits−1)) + log_val_accum( (num_stages−1)*num_freq_bits to ( (num_stages−
1)*num_freq_bits)+(num_freq_bits−1)) ;
          Pi_f <= log_val_accum( (num_stages−1)*num_freq_bits to (
(num_stages−1)*num_freq_bits)+(num_freq_bits−1));
      frequency_pipeline <= (others => ‘0’);
    case (currState) is
        when reset =>
          frequency <= (others => ‘0’);
          low_freq_range <= (others => ‘0’);
          frequency_pipeline <= (others => ‘0’);
          log_val_accum <= (others => ‘0’);
          logadder_pipe <= (others => ‘0’);
        when update_stats =>
          hist_max_freq_reg <= (others => ‘0’);
          for i in 0 to num_bins−1 loop
          saved_freq <= frequency;
            if evaluate_mass = ‘0’ then
              frequency( i*num_freq_bits to (i*num_freq_bits)
+ num_freq_bits−1 ) <= frequency( i*num_freq_bits to (i*num_freq_bits) + num_freq_bits−1 ) +
increment_amount(i*4 to (i*4)+3);
              log_val_accum <= (others => ‘0’);
            else
              for i in 0 to num_stages−1 loop
                saved_frequency_table <= frequency;
              end loop;
              frequency <= (others => ‘0’);
            end if;
          end loop;
        when locate_max_freq =>
          hist_max_freq <= hist_max_freq_reg;
          element_counter <= element_counter+1;
          if (saved_freq(0 to num_freq_bits−1) >= hist_max_freq_reg) then
            hist_max_freq_reg <= saved_freq(0 to num_freq_bits−1);
          end if;
          for i in 0 to num_bins−2 loop
```

| | |
|---|---|
| saved_freq(i*(num_freq_bits) | to |
| (i*(num_freq_bits)+num_freq_bits−1)) <= saved_freq((i+1)*(num_freq_bits) | to |

```
((i+1)*(num_freq_bits)+num_freq_bits−1) ) ;
          end loop;
        when rank_masses =>
          temp_test <= (others=> ‘0’);
          if evaluate_mass = ‘1’ then
            for i in 0 to num_stages−1 loop
            if matching_mass( (num_stages−1) − i) = ‘1’ then
              for j in 0 to num_bins−1 loop
                if increment_range( (i*num_bins) + j )
= ‘1’ then
                  logadder_pipe(
i*num_freq_bits to (i*num_freq_bits + (num_freq_bits−1)) ) <= saved_frequency_table( (127−j)*num_freq_bits to
((127−j)*num_freq_bits)+ (num_freq_bits−1));
-- temp_test( i*num_freq_bits
to (i*num_freq_bits + (num_freq_bits−1)) ) <= “01001101”;
                end if;
              end loop;
            end if;
            end loop;
          end if;
        when others =>
      end case;
    end if;
    end if;
  end process;
```

### FULL CITATIONS FOR PUBLICATIONS REFERRED TO IN THE SPECIFICATION

1. Choudary, Jyoti S., et al. “Interrogating the human genome using uninterpreted mass spectrometry data”, Proteomics, 1, pp. 651-667, 2001
2. Lesk, Arthur M Introduction to Bioinformatics. Oxford press, NY, 2002, pp. 6-7
3. Baxevais and Ouellette, Bioinformatics, Wiley Interscience, N, 2001, pp. 253-255
4. Taylor, J. Alex and Johnson, Richard S. “Implementation and Uses of Automated de Novo Peptide Sequencing by Tandem Mass Spectrometry”, Analytical Chemistry, 2001, V 73, pp 2594-2604
5. Eng, J. K, McCormack, A. L., and Yates, J. R., III, An approach to correlate tandem mass spectral data of peptides with amino acid sequences in a protein database. J. Am. Soc. Mass Spectrom., 5(11) 976-89 (1994)
6. Pappin, D. J. C., Hojrup, P. and Bleasby, A. J., Rapid identification of proteins by peptide mass fingerprinting. Curr Biol, 3(6) 327-32 (1993)
7. McLuckey, S. A. and Wells, J. M. “Mass Analysis at the Advent of the 21^{st} Century”, Chem Rev. 101 (2) (2001) pp. 571-606
8. Hellman, U., Wernstedt, C., Gonez, J. and Heldin, C. H. “Improvement of an “In-Gel” Digestion Procedure for the Micropreparation of Internal Protein Fragments for Amino Acid Sequencing” Analytical Biochemistry Volume: 224, Issue: 1, January 1995, pp. 451-455.
9. Washington University, Dept. of Chemistry, Instrumentation and Ionization Methods Tutorial http://wunmr.wustl.edu/˜msf/ionmethd.html
10. Caprioli, Richard and Sutter, Marc Mass Spectrometry, http://ms.mc.vanderbilt.edu/tutorials/ms/ms.htm
11. TM3 Documentation, University of Toronto, Dept. of ECE. http://www.eecg.toronto.edu/˜tm3/
12. Kumar, A., Harrison, P. M., et al, “An integrated approach for finding overlooked genes in yeast”, Nat Biotechnol. 2002 January; 20(1):27-8.
13. TM3 Ports Package Documentation, University of Toronto, Dept. of ECE. http://www.eecg.toronto.edu?tm3/ports.ps
14. Sinclair B., “Software Solutions to Proteomics Problems”, The Scientist, 2001 Oct., 15[20]:26
15. ftp://genome-ftp.stanford.edu/pub/yeast/data_download/sequence/genomic_sequence/chromosomes/fasta/
16. Partial Saccharomyces Chromosome IV map http://db.yeastgenome.org/cgi-bin/SGD/ORFMAP/ORFmap?seq=YDL229W
17. Partial Saccharomyces Chromosome XIV map http://db.yeastgenome.org/cgi-bin/SGD/ORFMAP/ORFmap?seq=YNL209W
18. Partial Saccharomyces Chromosome XV map http://db.yeastgenome.org/cgi-bin/SGD/ORFMAP/ORFmap?seq=YOR370C
19. BLAST (2 sequence) http://www.ncbi.nlm.nih.gov/blast/b12sec/b12.html
20. Sherman, Fred, An Introduction to the Genetics and Molecular Biology of the Yeast Saccharomyces cerevisiae, http://dbb.urmc.rochester.edu/labs/Sherman_f/yeast/index.html, Chapters 1-5
21. Stanchi F., Bertocco B., et al “Characterization of 16 novel human genes showing high similarity to yeast sequences”, Yeast. 2001 Jan. 15; 18(1), pp. 69-80.
22. MASCOT http://www.matrixscience.com/cgi/index.pl?page=/search_form_select.html
23. Net Gene Predictor http://www.cbs.dtu.dk/services/NetGene2/
24. GLIMMER at TIGR http://www.tigr.org/˜salzberg/glimmer.html
25. Houle, John L., “Database Mining in the Human Genome Initiative”, Whitepaper, Biodatabases, Amita Corporation, July 2000.
26. Altera Corporation, North American price list (volumes 100-499), August 2003
27. Leontti J., Private Communication, Camtech II Circuits, September 2003
28. Schaer, Steve, Personal Communication
29. Kingston Technology, http://www.kingston.com
30. Dell Computers http://www.dell.com
31. Xilinx Corporation http://www.xilinx.com
32. Altera Corporation http://www.altera.com
33. Ho, Yuen, Gruhler, Albrecht et al “Systematic identification of protein complexes in Saccharomyces cerevisiae by mass spectrometry”, Nature 2002 Jan. 10; 415(6868): 180-183
34. Stratix power calculator, http://www.altera.com/products/devices/stratix/utilities/power_calculator/stratix_power_calc.xls
35. Sonar MS/MS, http://www.genomicsolutions.com/search/index.html
36. ThermoFinnigan Sequest, http://www.genomicsolutions.com/search/index.html
37. MDS Proteomics Pepsea, http://www.mdsproteomics.com

1. A method for identifying a protein through amino acid sequences of one or more query peptides generated from the protein comprising:
(a) translating amino acid sequences of one or more query peptides to all possible codons from which the peptides can be synthesized to prepare strings of codons;
(b) searching known nucleic acid sequences to locate one or more known nucleic acids that comprise regions that match the strings of codons; and
(c) ranking two or more matching nucleic acids to identify nucleic acids that are true coding regions for the protein to thereby identify the protein.

2. A method of claim 1 wherein the amino acid sequences of the query peptides are obtained from the spectra produced by mass spectrometry of the peptides.

3. A method of claim 1 wherein in (b) the searching comprises simultaneously providing the strings of codons as parallel queries to a database of known nucleic acid sequences.

4. A method of claim 1 wherein in (b) the searching further comprises locating one or more known nucleic acids that comprise regions that match reverse complements of the strings of codons.

5. A method of claim 1 wherein in (c) the ranking is based on a comparison of masses of peptides translated from sequences in proximity to the regions in the known nucleic acids that match the strings of codons with masses of peptides of the protein other than the query peptides.

6. A method of claim 1 wherein the strings of codons comprise wildcards.

7. A method of claim 1 wherein in (c) the ranking comprises the following steps:
(a) calculating the masses of peptides translated from sequences in proximity to the regions in the known nucleic acids that match the strings of codons;
(b) comparing the masses calculated in (a) with masses of peptides of the protein other than the query peptides, or fragments thereof, to identify peptides with matching masses;
(c) assigning scores to each matching mass and accumulating the scores for all matching masses in proximity to the regions in the known nucleic acids that match strings of codons; and
(d) ranking two or more nucleic acids that match the strings of codons based on the accumulated scores to identify potential nucleic acids encoding the protein to thereby identify the protein.

8. A method of claim 7 wherein in (b) the masses of peptides of the protein other than the query peptides or fragments thereof are identified through mass spectrometry.

9. A method of claim 8 wherein the masses of the peptides are identified in a precursor ion scan.

10. A computer implemented system for identifying a protein through amino acid sequences of one or more query peptides generated from the protein comprising:
(a) a search engine for locating regions of known nucleic acid sequences that match strings of codons translated from one or more query peptides;
(b) a mass calculator for calculating masses of peptides translated from sequences in proximity to regions in known nucleic acid sequences that match the strings of codons;
(c) optionally a scoring unit for (i) comparing masses calculated in (b) with masses of peptides of the protein other than the query peptides to identify peptides with matching masses; (ii) assigning scores to peptides with matching masses; and (iii) accumulating scores for all matching masses in proximity to or around the regions located in (a) to evaluate the likelihood that a region is a true coding region for the protein.

11. A method for identifying a protein comprising:
(a) providing amino acid sequences of peptides generated by mass spectrometry of the peptides cleaved from the protein;
(b) translating amino acid sequences of one or more query peptides to all possible codons from which the peptides can be synthesized to prepare strings of codons;
(c) searching known nucleic acid sequences to locate one or more known nucleic acids that comprise regions that match the strings of codons; and
(d) optionally ranking two or more matching nucleic acids located in (c) by
(i) calculating the masses of peptides translated from sequences in proximity to regions in the known nucleic acids that match the strings of codons;
(ii) comparing the masses calculated in (i) with masses identified by mass spectrometry for peptides of the protein other than the query peptides to identify peptides with matching masses;
(iii) assigning scores to each matching mass and accumulating the scores for all matching masses in proximity to regions in known nucleic acids that match the strings of codons; and
(iv) ranking two or more known nucleic acids that match the strings of codons based on the accumulated scores to identify potential nucleic acids encoding the protein to thereby identify the protein.

12. A method of claim 1 wherein the query peptides are tryptic peptides.

13. A programmable hardware employing a method as claimed in claim 1.

14. A hardware acceleration system for identification of a protein comprising a generic circuit board capable of being plugged into a computing device wherein the circuit board comprises logic chips and memory wherein the memory comprises nucleic acid sequence information, and the chips provide means to search through the nucleic acid sequence information for regions matching strings of codons translated from one or more query peptides provided as input to the computing device.

15. A hardware acceleration system for identification of a protein comprising a generic circuit board capable of being plugged into a computing device wherein the circuit board comprises logic chips and memory wherein the memory comprises nucleic acid sequence information, and the chips provide means to search through the nucleic acid sequence information for patterns matching a query that has been provided to the computing device as input from a mass spectrometer.

16. A method as claimed in claim 1 implemented using field programmable gate array (FPGA) technology.

17. A method as claimed in claim 1 implemented using application-specific integrated circuit (ASIC) technology.

18. A method as claimed in claim 1 wherein known nucleic acid sequences comprise a genome, in particular human genome.

19. A database comprising a set of masses corresponding to the masses of the query peptides and the peptides translated from a matching region in proximity to or around a known nucleic acid generated in accordance with a method of claim 1.

20. Computerized representations of information generated using a method of any preceding claim, including any electronic, magnetic, or electromagnetic storage forms of the information needed to define it such that the information will be computer readable for purposes of display and/or manipulation.

21. A computer comprising a machine-readable data storage medium comprising a data storage material encoded with machine readable data wherein said data comprises information generated using a method of claim 1.

22. A method for presenting information pertaining to nucleic acids that potentially encode a protein the method comprising the steps of:
(a) providing an interface for entering query information generated from mass spectrometry relating to amino acid sequences of peptides generated or cleaved from the protein;
(b) examining records in a database of known nucleic acid sequences to locate regions in the nucleic acid sequences matching strings of codons translated from the entered query peptides' amino acid sequence information;
(c) displaying the data relating to the matched string of codons and regions in the nucleic acids; and
(d) optionally displaying the masses of the peptides generated from mass spectrometry and the masses of peptides encoding regions in proximity to the regions of known nucleic acids that match the string of codons.

23. A computer program product comprising a computer-usable medium having computer-readable program code embodied thereon for effecting the steps of a method of claim 1.

24. A method of using a method, system, programmable hardware, database, product, or computer as claimed in claim 1 to identify proteins associated with disease or that can be used in drug design.

25. A method of using a method, system, programmable hardware, database, product, or computer as claimed in claim 1 to identify proteins in samples from patients.
